SEQUENCE LISTING (28 sequences)

SEQ ID NO: 1 - DNA, 257 bp, Artificial
  Synthetic bAS3 intron 2 sequence (5′-3′)
gtgagtctatgggacccttgatgttttctttccccttcttttctatggttaagttcatgtcataggaaggggagaagtaacagggtatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 2 - DNA, 21 bp, Artificial
  Synthetic guide strand-shRNA BCL11A-XL
gcgcgatcgagtgttgaataa

SEQ ID NO: 3 - DNA, 15 bp, Artificial
  Synthetic loop segment
ctccatgtggtagag

SEQ ID NO: 4 - DNA, 57 bp, Artificial
  Synthetic shRNA BCL11A-XL
gcgcgatcgagtgttgaataactccatgtggtagagttattcaacactcgatcgcgc

SEQ ID NO: 5 - DNA, 104 bp, Artificial
  Synthetic amiR-shRNA BCL11A-XL
cctggcctcctgcagtgccacgctgcgcgatcgagtgttgaataactccatgtggtagagttattcaacactcgatcgcgcagtgcggcacatgcttaccagct

SEQ ID NO: 6 - DNA, 361 bp, Artificial
  Synthetic bAS3-miR/int2_del/amiR-shRNA BCL11A-XL
gtgagtctatgggacccttgatgttttctttccccttcttttctatggttaagttcatgtcataggaaggggagaagtaacagggcctggcctcctgcagtgccacgctgcgcgatcgagtgttgaataactccatgtggtagagttattcaacactcgatcgcgcagtgcggcacatgcttaccagcttatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 7 - DNA, 361 bp, Artificial
  Synthetic bAS3-miR/int2/amiR-shRNA BCL11A-XL
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctgcgcgatcgagtgttgaataactccatgtggtagagttattcaacactcgatcgcgcagtgcggcacatgcttaccagcttccagctaccattctgcttttattttatggttgggataaggctggattattctgagtccaagctaggcccttttgctaatcatgttcatacctcttatcttcctcccacag

SEQ ID NO: 8 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 9 - DNA, 2051 bp, Artificial
  Synthetic bAS3 sequence (5′-3′) + ( bAS3-miR/int2_del/amiR-shRNABCL11A-XL)
acatttgcttctgacacaactgtgttcactagcaacctcaaacagacaccatggtgcacctgactcctgaggagaagtctgccgttactgccctgtgggacaaggtgaacgtggatgccgttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgcccagctgagtgagctgcactgtgacaagctgcacgtggatcctgagaacttcagggtgagtctatgggacccttgatgttttctttccccttcttttctatggttaagttcatgtcataggaaggggagaagtaacagggcctggcctcctgcagtgccacgctgcgcgatcgagtgttgaataactccatgtggtagagttattcaacactcgatcgcgcagtgcggcacatgcttaccagcttatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatggtacgtaccgataaaattttgaattttgtaatttgtttttgtaattctttagtttgtatgtctgttgctattatgtctactattctttcccctgcactgtaccccccaatccccccttttcttttaaaagttaaccgataccgtcgagatccgttcactaatcgaatggatctgtctctgtctctctctccaccttcttcttctattccttcgggcctgtcgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagcttgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatccttt

SEQ ID NO: 10 - DNA, 2051 bp, Artificial
  Synthetic bAS3 sequence (5′-3′) + (bAS3-miR/int2/amiR-shRNA BCL11A-XL)
acatttgcttctgacacaactgtgttcactagcaacctcaaacagacaccatggtgcacctgactcctgaggagaagtctgccgttactgccctgtgggacaaggtgaacgtggatgccgttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgcccagctgagtgagctgcactgtgacaagctgcacgtggatcctgagaacttcagggtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctgcgcgatcgagtgttgaataactccatgtggtagagttattcaacactcgatcgcgcagtgcggcacatgcttaccagcttccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatggtacgtaccgataaaattttgaattttgtaatttgtttttgtaattctttagtttgtatgtctgttgctattatgtctactattctttcccctgcactgtaccccccaatccccccttttcttttaaaagttaaccgataccgtcgagatccgttcactaatcgaatggatctgtctctgtctctctctccaccttcttcttctattccttcgggcctgtcgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagcttgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatccttt

SEQ ID NO: 11 - DNA, 26 bp, Artificial
  Synthetic probe
cctgtcatgcccacacaaatctctcc

SEQ ID NO: 12 - DNA, 22 bp, Artificial
  Synthetic primer
gctgtcatctcttgtgggctgt

SEQ ID NO: 13 - DNA, 21 bp, Artificial
  Synthetic primer
actcatgggagctgctggttc

SEQ ID NO: 14 - DNA, 19 bp, Artificial
  Synthetic probe
cgcacggcaagaggcgagg

SEQ ID NO: 15 - DNA, 20 bp, Artificial
  Synthetic primer
tcccccgcttaatactgacg

SEQ ID NO: 16 - DNA, 20 bp, Artificial
  Synthetic primer
caggactcggcttgctgaag

SEQ ID NO: 17 - DNA, 22 bp, Artificial
  Synthetic primer
gccaccactttctgataggcag

SEQ ID NO: 18 - DNA, 18 bp, Artificial
  Synthetic primer
aagggcacctttgcccag

SEQ ID NO: 19 - DNA, 20 bp, Artificial
  Synthetic primer
atgcgagctgtgcaactatg

SEQ ID NO: 20 - DNA, 20 bp, Artificial
  Synthetic primer
gtaaacgtccttccccacct

SEQ ID NO: 21 - DNA, 19 bp, Artificial
  Synthetic primer
cctgtcctctgcctctgcc

SEQ ID NO: 22 - DNA, 19 bp, Artificial
  Synthetic primer
ggattgccaaaacggtcac

SEQ ID NO: 23 - DNA, 20 bp, Artificial
  Synthetic primer
agttcacgcccaagtacatc

SEQ ID NO: 24 - DNA, 19 bp, Artificial
  Synthetic primer
cttcacagtcctcatggcc

SEQ ID NO: 25 - DNA, 21 bp, Artificial
  Synthetic primer
cggtcaacttcaagctcctaa

SEQ ID NO: 26 - DNA, 20 bp, Artificial
  Synthetic primer
acagaagccaggaacttgtc

SEQ ID NO: 27 - RNA, 104 nt, Artificial
  Synthetic amiR sequence of Figure 1A
  misc_feature (25)..(45): n is a, c, g, or u
  misc_feature (61)..(81): n is a, c, g, or u
ccuggccuccugcagugccacgcunnnnnnnnnnnnnnnnnnnnncuccaugugguagagnnnnnnnnnnnnnnnnnnnnnagugcggcacaugcuuaccagcu

SEQ ID NO: 28 - RNA, 104 nt, Artificial
  Synthetic amiR sequence of Figure 1B
ccuggccuccugcagugccacgcugcgcgaucgaguguugaauaacuccaugugguagaguuauucaacacucgaucgcgcagugcggcacaugcuuaccagcu

(19) United States
(12) Patent Application Publication
(10) Pub. No.: US 2022/0160788 A1
(43) Pub. Date: May 26, 2022

(54) BIFUNCTIONAL VECTORS ALLOWING BCL11A SILENCING AND EXPRESSION OF AN ANTI-SICKLING HBB AND USES THEREOF FOR GENE THERAPY OF B-HEMOGLOBINOPATHIES

(71) Applicants: INSERM (INSTITUT NATIONAL DE LA SANTÉ ET DE LA RECHERCHE MÉDICALE), Paris (FR); UNIVERSITÉ D'EVRY-VAL-D'ESSONNE, Evry (FR); ASSISTANCE PUBLIQUE-HÔPITAUX DE PARIS (APHP), Paris (FR); UNIVERSITÉ DE PARIS, Paris (FR); FONDATION IMAGINE, Paris (FR)

(72) Inventors: Annarita MICCIO, Paris (FR); Mario AMENDOLA, Evry (FR); Mégane BRUSSON, Paris (FR); Marina CAVAZZANA, Paris (FR); Fulvio MAVILIO, Modena (IT)

(21) Appl. No.: 17/441,466

(22) PCT Filed: Mar. 20, 2020
(86) PCT No.: PCT/EP2020/057876

§ 371 Date: Sep. 21, 2021

(30) Foreign Application Priority Data
Mar. 22, 2019 (EP) 19305356.8

(51) Int. Cl. A61K 35/545; C12N 15/86; C12N 15/113; C12N 5/0735; A61K 35/28; A61K 38/17; A61P 7/06

(52) U.S. Cl. CPC A61K 35/545; C12N 15/86; C12N 15/113; C12N 5/0606; A61K 35/28; A61K 48/00; A61P 7/06; C12N 2310/531; C12N 2310/141; C12N 2740/15043; C12N 2310/14; A61K 38/1722

(57) ABSTRACT

The #β-hemoglobinopathies #β-thalassemia (BT) and sickle cell disease (SCD) are the most frequent genetic disorders worldwide. These diseases are caused by mutations causing reduced or abnormal synthesis of the β-globin chain of the adult hemoglobin (Hb) tetramer. Here, the inventors intend to improve HSC-based gene therapy for β-thalassemia and SCD by developing an innovative, highly infectious LV vector expressing a potent anti-sickling β-globin transgene and a second biological function either increasing fetal γ-globin expression (for β-thalassemia and SCD). More particularly, the inventors have designed a novel lentivirus (LV), which carry two different functions: βAS3 gene addition and gene silencing. This last strategy allows the re-expression of the fetal γ-globin genes (HBG1 and HBG2) and production of the endogenous fetal hemoglobin (HbF). Elevated levels of HbF and HbAS3 (Hb tetramer containing βAS3-globin) will benefit the β-hemoglobinopathy phenotype by increasing the total amount of β-like globin that will: (i) reduce the alpha precipitates and improve the alpha/non alpha ratio in β-thalassemia, and (ii) reduce the sickling in SCD. This combined strategy will improve the β-hemoglobinopathy phenotype at a lower vector copy number (VCN) per cell compared to a LV expressing the βAS3 alone.

### FIELD OF THE INVENTION

The present invention relates to bifunctional vectors allowing BCL11A silencing and expression of an anti-sickling HBB and uses thereof for gene therapy of β-hemoglobinopathies.

### BACKGROUND OF THE INVENTION

The β-hemoglobinopathies β-thalassemia (BT) and sickle cell disease (SCD) are the most frequent genetic disorders worldwide. These diseases are caused by mutations causing reduced or abnormal synthesis of the β-globin chain of the adult hemoglobin (Hb) tetramer.

β-thalassemia (BT) is a genetic disorder with an estimated annual incidence of 1:100,000 worldwide and 1:10,000 in Europe. This disease is caused by more than 200 mutations (mainly point mutations) localized in functionally important regions of the β-globin (HBB) gene. The total absence of the β-globin chain (β0) is usually associated with the most severe clinical phenotype. Reduced or absent β-globin chain production is responsible for precipitation of uncoupled α-globin chains, which in turn leads to erythroid precursor apoptosis and impairment in erythroid differentiation (i.e. ineffective erythropoiesis), and hemolytic anemia.

Sickle cell disease (SCD) is a severe genetic disorder affecting ˜312,000 newborns worldwide annually. A single point mutation in the HBB gene causes a Glu>Val amino acid substitution in the β-globin chain (β^{S}-globin). The sickle hemoglobin (HbS, α₂β^{S}₂) has the propensity to polymerize under deoxygenated conditions, resulting in the production of sickle-shaped red blood cells (RBCs) that cause occlusions of small blood vessels, leading to impaired oxygen delivery to tissues, multiple organ damage, severe pain and early mortality.

Symptomatic treatment of β-hemoglobinopathies (e.g., RBC transfusions and supportive care) are associated with high costs, reduced life expectancy and poor quality of life. The only curative option is allogeneic transplantation of hematopoietic stem cells (HSC), which, however, is severely limited by the availability of compatible donors.

Transplantation of autologous HSC corrected by lentiviral (LV) vectors expressing a β-globin transgene is a promising therapeutic option. However, this treatment is at best partially effective in correcting the clinical phenotype in patients with severe β-thalassemia or SCD. Hence, despite the undeniable progress in the field of gene therapy, the treatment of these blood diseases requires further key improvements. Firstly, greater Hb production per cell is required—especially for severe forms of β-thalassemia (e.g. β⁰/β⁰ patients with no residual expression of the β-globin gene) and SCD (where high expression of antisickling globin will favor its incorporation into Hb, at the expense of the sickle β-globin). Secondly, reduced expression of the sickle β-globin gene (in SCD) is an important goal because elevated HbS levels are associated with a greater incidence of vaso-occlusive crises.

The inventors had previously designed a high-titer LV for β-globin expression termed GLOBE (Miccio et al., 2011, 2008), which is currently in clinical trial for β-thalassemia at the San Raffaele Hospital in Milan (Marktel et al., 2019). They have recently adapted the GLOBE vector to gene therapy of SCD by introducing 3 anti-sickling mutations in the β-globin gene that impair HbS polymerization (βAS3 LV) (Weber et al., 2018). Although the inventors obtained high LV copy number in hematopoietic stem/progenitor cells (HSPC) derived from a SCD patient, the RBC phenotype was only partially corrected, indicating that a classical gene addition strategy is hampered by the high level of the endogenous β^{S}-globin expression that is not sufficiently competed by the anti-sickling βAS3 (Weber et al., 2018). Therefore, additional improvements in LV design are required to obtain a robust therapeutic correction of the β-thalassemic and SCD severe clinical phenotypes.

### SUMMARY OF THE INVENTION

As defined by the claims, the present invention relates to bifunctional vectors allowing BCL11A silencing and expression of an anti-sickling HBB and uses thereof for gene therapy of β-hemoglobinopathies.

### DETAILED DESCRIPTION OF THE INVENTION

Here, the inventors intend to improve HSC-based gene therapy for β-thalassemia and SCD by developing an innovative, highly infectious LV vector expressing a potent anti-sickling β-globin transgene and a second biological function either increasing fetal γ-globin expression (for β-thalassemia and SCD). More particularly, the inventors have designed a novel lentivirus (LV), which carry two different functions: βAS3 gene addition and gene silencing. This last strategy allows the re-expression of the fetal γ-globin genes (HBG1 and HBG2) and production of the endogenous fetal hemoglobin (HbF). Elevated levels of HbF and HbAS3 (Hb tetramer containing βAS3-globin) will benefit the β-hemoglobinopathy phenotype by increasing the total amount of β-like globin that will: (i) reduce the alpha precipitates and improve the alpha/non alpha ratio in β-thalassemia, and (ii) reduce the sickling in SCD. This combined strategy will improve the β-hemoglobinopathy phenotype at a lower vector copy number (VCN) per cell compared to a LV expressing the βAS3 alone.

The first object of the present invention relates to a nucleic acid molecule having the sequence as set forth in SEQ ID NO:1 wherein a sequence encoding for an artificial microRNA (amiR) suitable for reducing the expression of BCL11A (in particular of the BCL11A-XL isoform) is inserted i) between the nucleotide at position 85 and the nucleotide 86 at position in SEQ ID NO:1 and/or ii) between the nucleotide at position 146 and the nucleotide 147 at position in SEQ ID NO:1.

SEQ ID NO: 1
>bAS3 intron 2 sequence (5′-3′)
gtgagtctatgggacccttgatgttttctttccccttcttttctatggtta
agttcatgtcataggaaggggagaagtaacagggtatttctgcatataaat
tgtaactgatgtaagaggtttcatattgctaatagcagctacaatccagct
accattctgcttttattttatggttgggataaggctggattattctgagtc
caagctaggcccttttgctaatcatgttcatacctcttatcttcctcccac
ag

As used herein, the term “BCL11A” has its general meaning in the art and refers to the gene encoding for BAF chromatin remodeling complex subunit BCL11A (Gene ID: 53335). The term is also known as EVI9; CTIP1; DILOS; ZNF856; HBFQTL5; BCL11A-L; BCL11A-S; BCL11a-M; or BCL11A-XL. Five alternatively spliced transcript variants of this gene, which encode distinct isoforms, have been reported. The protein associates with the SWI/SNF complex that regulates gene expression via chromatin remodelling. BCL11A is highly expressed in several hematopoietic lineages, and plays a role in the switch from γ- to β-globin expression during the fetal to adult erythropoiesis transition (Sankaran V J et al. “Human fetal hemoglobin expression is regulated by the developmental stage-specific repressor BCL11A”, Science Science. 2008 Dec 19;322(5909): 1839-42).

As used herein, the term “microRNA”, “miRNA” or “miR” has its general meaning in the art and refers to a small non-coding RNA molecule (containing about 22 nucleotides) found in plants, animals and some viruses, that functions in RNA silencing and post-transcriptional regulation of gene expression. miRNAs resemble the small interfering RNAs (siRNAs) of the RNA interference (RNAi) pathway, except that miRNAs derive from regions of RNA transcripts that fold back on themselves to form short hairpins, whereas siRNAs derive from longer regions of double-stranded RNA. The miRNAs are first transcribed as primary miRNAs (pri-miRNAs) with caps and a poly-A tail. The pri-miRNAs are then processed into precursor miRNAs (pre-miRNAs) by an enzyme called Drosha. The structure of pre-miRNA is a 70 nucleotide-long stem-loop structure. The pre-miRNAs are then exported into the cytoplasm and split into mature miRNAs by an enzyme called Dicer. These mature miRNAs will integrate into the RNA-induced silencing complex (RISC) and activate the RISC. The activated RISC can then allow miRNAs to bind with the targeted mRNA and silence the gene expression.

As used herein, the term “artificial miRNA”, “artificial miR” or “amiR” refers to a shRNA that is embedded into a miRNA backbone that is derived from a naturally-occurring miRNA. More particularly, the amiR of the present invention consists of a shRNA having 5′ and 3′flanking regions with one or more structural features of a corresponding region of a naturally-occurring miRNA. For example, any miRNAs described in miRBase can be used for providing the miRNA backbone.

In some embodiments, the miRNA backbone is derived from miR-142, miR-155, miR-181 and miR-223.

As used herein, the term “miR-142” has its general meaning in the art and refers to the miR available from the data base http://mirbase.org under the miRBase accession number MI0000458 (hsa-mir-142).

As used herein, the term “miR-155” has its general meaning in the art and refers to the miR available from the data base http://mirbase.org under the miRBase accession number MI0000681 (hsa-mir-155).

As used herein, the term “miR-181” has its general meaning in the art and refers to the miR available from the data base http://mirbase.org under the miRBase accession number MI0000289 (hsa-mir-181).

As used herein, the term “miR-223” has its general meaning in the art and refers to the miR available from the data base http://mirbase.org under the miRBase accession number MI0000300 (hsa-mir-223).

Typically, the structure of the amiR of the present invention is depicted in FIGS. 1A & 1B. Mechanistically, the artificial miRNA is first cleaved to produce the shRNA and then cleaved again by DICER to produce siRNA. The siRNA is then incorporated into the RISC for target mRNA degradation.

As used herein, the term “short hairpin RNA” or “shRNA” has its general meaning in the art and refers to a unimolecular RNA that is capable of performing RNA interference and that has a passenger strand, a loop, and a guide strand. Typically, the shRNA of the present invention adopts a stem-loop structure. As used herein, a “stem-loop structure” refers to a nucleic acid having a secondary structure that includes a region of nucleotides which are known or predicted to form a double strand or duplex (stem portion or stem region) that is linked on one side by a region of predominantly single-stranded nucleotides (loop portion or terminal loop region). The terms “hairpin” and “fold-back” structures can also be used to refer to stem-loop structures. Such structures are well known in the art and the term is used consistently with its known meaning in the art. As described herein, the stem region is a region formed by a guide strand and a passenger strand. As described herein, the “guide strand” represents the portion that associates with RISC as opposed to the “passenger strand”, which is not associated with RISC. Typically, the passenger and guide strands are thus substantially complementary to each other. The passenger/guide strand can be about 11 to about 29 nucleotides in length, and more preferably 17 to 19 nucleotides in length.

In some embodiments, the sequence encoding for the guide strand consists of the sequence as set forth in SEQ ID NO: 2.

SEQ ID NO: 2
>(guide strand-shRNA BCL11A-XL)
GCGCGATCGAGTGTTGAATAA

In some embodiments, the guide strand that is complementary to the target can contain mismatches. In some embodiments, the guide strand and the passenger strand may have at least one base pair mismatch. In some embodiments, the guide strand and the passenger strand have 2 base pair mismatches, 3 base pair mismatches, 4 base pair mismatches, 5 base pair mismatches, 6 base pair mismatches, 7 base pair mismatches, 8 base pair mismatches, 9 base pair mismatches, 10 base pair mismatches, 11 base pair mismatches, 12 base pair mismatches, 13 base pair mismatches, 14 base pair mismatches or 15 base pair mismatches. In some embodiments, the guide strand and passenger strand have mismatches at no more than ten consecutive base pairs. In some embodiments, at least one base pair mismatch is located at an anchor position. In some embodiments, at least one base pair mismatch is located in a center portion of the stem.

As described herein, the terminal loop region comprises at least 4 nucleotides. The sequence of the loop can include nucleotide residues unrelated to the target. In some embodiments, the loop segment is encoded by the sequence as set forth in SEQ ID NO:3.

SEQ ID NO: 3
>(loop segment)
CTCCATGTGGTAGAG

In some embodiments, the sequence encoding for the shRNA of the present invention is sequence SEQ ID NO:4. The loop of the shRNA is framed.

(shRNA BCL11A-XL)
>SEQ ID NO: 4

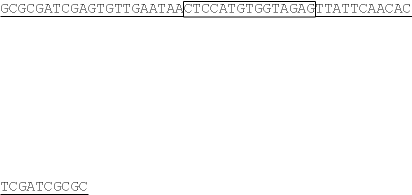

In some embodiments, the sequence encoding for the amiR of the present invention is sequence SEQ ID NO:5 wherein the sequence of shRNA is underlined and the loop of the amiR is framed.

(amiR-shRNA BCL11A-XL)
>SEQ ID NO: 5

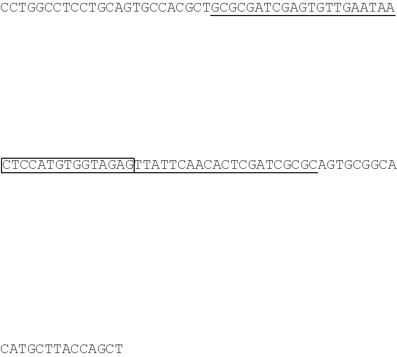

In some embodiments, the nucleic acid molecule of the present invention has a sequence as set forth in SEQ ID NO:6 or SEQ ID NO:7 wherein the 5′ to 3′ sequence of intron 2 of the βAS3 transgene are in lowercase, the amiR sequence is in uppercase, the sequence of shRNA is underlined and the loop of the amiR is framed.

(βAS3-miR/int2_del/amiR-shRNA BCL11A-XL)
>SEQ ID NO: 6
gtgagtctatgggacccttgatgttttctttccccttcttttctatggttaagttcatgtcataggaag

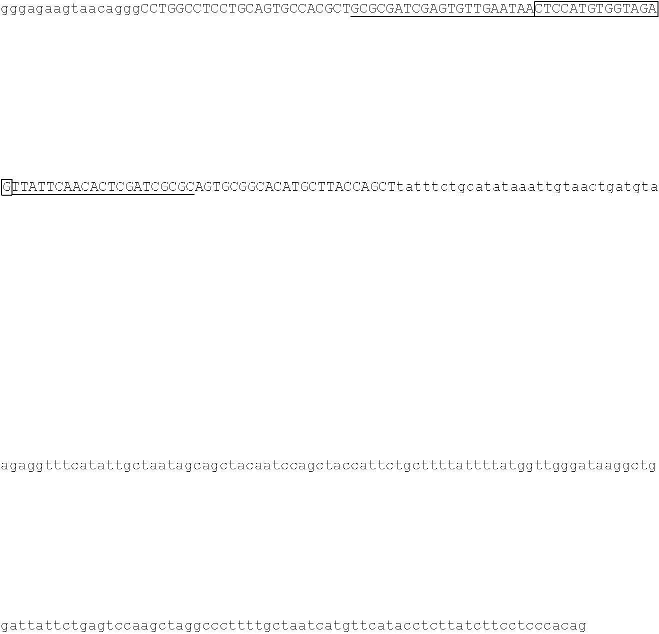

(βAS3-miR/int2/amiR-shRNA BCL11A-XL)
>SEQ ID NO: 7
gtgagtctatgggacccttgatgttttctttccccttcttttctatggttaagttcatgtcataggaag
gggagaagtaacagggtatttctgcatataaattgtaactgatgtaagaggtttcatattgctaatagcagctac

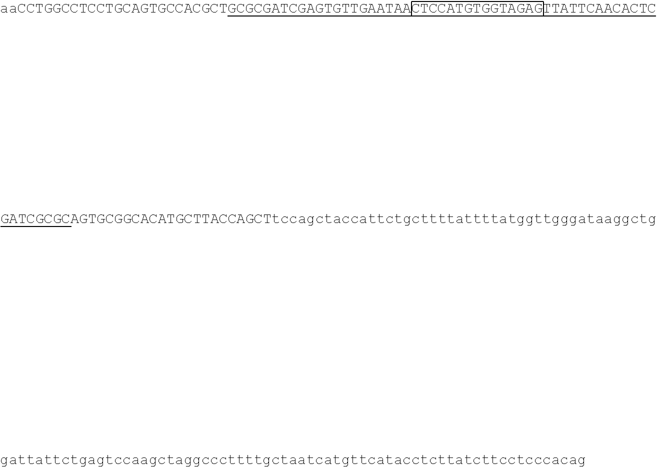

A further object of the present invention relates to a transgene encoding for an anti-sickling HBB, wherein said transgene comprises the nucleic acid molecule of the present invention.

As used herein, the term “β-globin” or “HBB” has its general meaning in the art and refers to a globin protein, which along with alpha globin (HBA), makes up the most common form of haemoglobin (Hb) in adult humans. Normal adult human Hb is a heterotetramer consisting of two alpha chains and two beta chains. HBB is encoded by the HBB gene on human chromosome 11. It is 146 amino acids long and has a molecular weight of 15,867 Da. An exemplary human amino acid sequence is represented by SEQ ID NO:8.

SEQ ID NO: 8
>sp|P68871|HBB_HUMAN Hemoglobin subunit beta OS =
Homo sapiens OX = 9606 GN = HBB PE = 1 SV = 2
MVHLTPEEKSAVTALWGKVNVDEVGGEALGRLLVVYPWTQRFFESFGDLST
PDAVMGNPKVKAHGKKVLGAFSDGLAHLDNLKGTFATLSELHCDKLHVDPE
NFRLLGNVLVCVLAHHFGKEFTPPVQAAYQKVVAGVANALAHKYH

As used herein, the term “hemoglobin S” or “HbS” has its general meaning in the art and refers to the mutated beta-globin encoded by the mutated sickle HBB gene. In SCD, hemoglobin S replaces both beta-globin subunits in hemoglobin. Typically, the mutation corresponds to E6V mutation wherein the amino acid glutamic acid is replaced with the amino acid valine at position 6 in beta-globin.

As used herein, the term “anti-sickling HBB” or “βAS3” refers to a HBB polypeptide that contains three mutations causing three potentially beneficial “anti-sickling” amino-acidic substitutions G16D, E22A, T87Q. Mutation E22A and T87Q impair, respectively, the axial and lateral contacts necessary for the formation of HbS polymers, and mutation G16D increases the affinity to HBA chains, thus conferring to βAS3 a competitive advantage for the incorporation in the Hb tetramers.

As used herein, the term “transgene” refers to any nucleic acid that shall be expressed in a mammal cell.

In some embodiments, the transgene of the present invention relates to the transgene described in Weber, L., et al. “An optimized lentiviral vector efficiently corrects the human sickle cell disease phenotype.” Molecular Therapy Methods & Clinical Development 10 (2018): 268-280, wherein intron 2 sequence is substituted by the nucleic acid molecule of the present invention (e.g. SEQ ID NO:6 or SEQ ID NO:7).

In some embodiments, the transgene comprises the sequence as set forth in SEQ ID NO:9 or SEQ ID NO:10.

>βAS3 sequence (5′-3′) + (βAS3-miR/int2_del/amiR-shRNA BCL11A-XL):
SEQ ID NO: 9
acatttgcttctgacacaactgtgttcactagcaacctcaaacagacaccatggtgcacctgactcctg
aggagaagtctgccgttactgccctgtgggacaaggtgaacgtggatgccgttggtggtgaggccctgg
gcaggttggtatcaaggttacaagacaggtttaaggagaccaatagaaactgggcatgtggagacagag
aagactcttgggtttctgataggcactgactctctctgcctattggtctattttcccacccttaggctg
ctggtggtctacccttggacccagaggttctttgagtcctttggggatctgtccactcctgatgctgtt
atgggcaaccctaaggtgaaggctcatggcaagaaagtgctcggtgcctttagtgatggcctggctcac
ctggacaacctcaagggcacctttgcccagctgagtgagctgcactgtgacaagctgcacgtggatcct

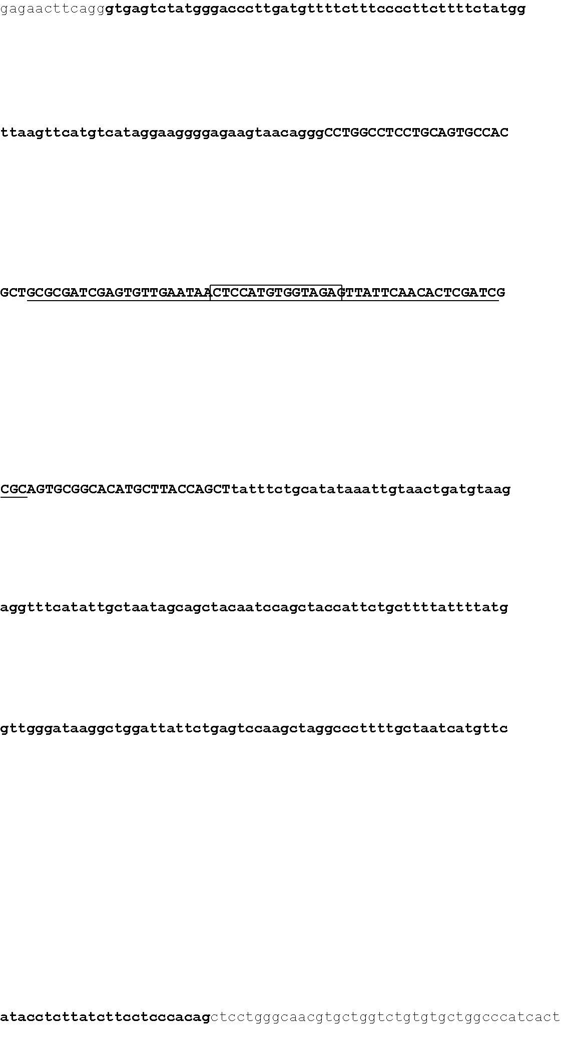

ttggcaaagaattcaccccaccagtgcaggctgcctatcagaaagtggtggctggtgtggctaatgccc
tggcccacaagtatcactaagctcgctttcttgctgtccaatttctattaaaggttcctttgttcccta
agtccaactactaaactgggggatattatgaagggccttgagcatctggattctgcctaataaaaaaca
tttattttcattgcaatgatgtatttaaattatttctgaatattttactaaaaagggaatgtgggaggt
cagtgcatttaaaacataaagaaatgaagagctagttcaaaccttgggaaaatacactatatcttaaac
tccatgaaagaaggtgaggctgcaaacagctaatgcacattggcaacagcccctgatgcctatgcctta
ttcatccctcagaaaaggattcaagtagaggcttgatttggaggttaaagttttgctatgctgtatttt
acattacttattgttttagctgtcctcatggtacgtaccgataaaattttgaattttgtaatttgtttt
tgtaattctttagtttgtatgtctgttgctattatgtctactattctttcccctgcactgtacccccca
atccccccttttcttttaaaagttaaccgataccgtcgagatccgttcactaatcgaatggatctgtct
ctgtctctctctccaccttcttcttctattccttcgggcctgtcgggtcccctcggggttgggaggtgg
gtctgaaacgataatggtgaatatccctgcctaactctattcactatagaaagtacagcaaaaactatt
cttaaacctaccaagcctcctactatcattatgaataattttatataccacagccaatttgttatgtta
aaccaattccacaaacttgcccatttatctaattccaataattcttgttcattcttttcttgctggttt
tgcgattcttcaattaaggagtgtattaagcttgtgtaattgttaatttctctgtcccactccatccag
gtcgtgtgattccaaatctgttccagagatttattactccaactagcattccaaggcacagcagtggtg
caaatgagttttccagagcaaccccaaatccccaggagctgttgatccttt
>βAS3 sequence (5′-3′) + (βAS3-miR/int2/amiR-shRNA BCL11A-XL):
SEQ ID NO: 10
acatttgcttctgacacaactgtgttcactagcaacctcaaacagacaccatggtgcacctgactcctg
aggagaagtctgccgttactgccctgtgggacaaggtgaacgtggatgccgttggtggtgaggccctgg
gcaggttggtatcaaggttacaagacaggtttaaggagaccaatagaaactgggcatgtggagacagag
aagactcttgggtttctgataggcactgactctctctgcctattggtctattttcccacccttaggctg
ctggtggtctacccttggacccagaggttctttgagtcctttggggatctgtccactcctgatgctgtt
atgggcaaccctaaggtgaaggctcatggcaagaaagtgctcggtgcctttagtgatggcctggctcac
ctggacaacctcaagggcacctttgcccagctgagtgagctgcactgtgacaagctgcacgtggatcct

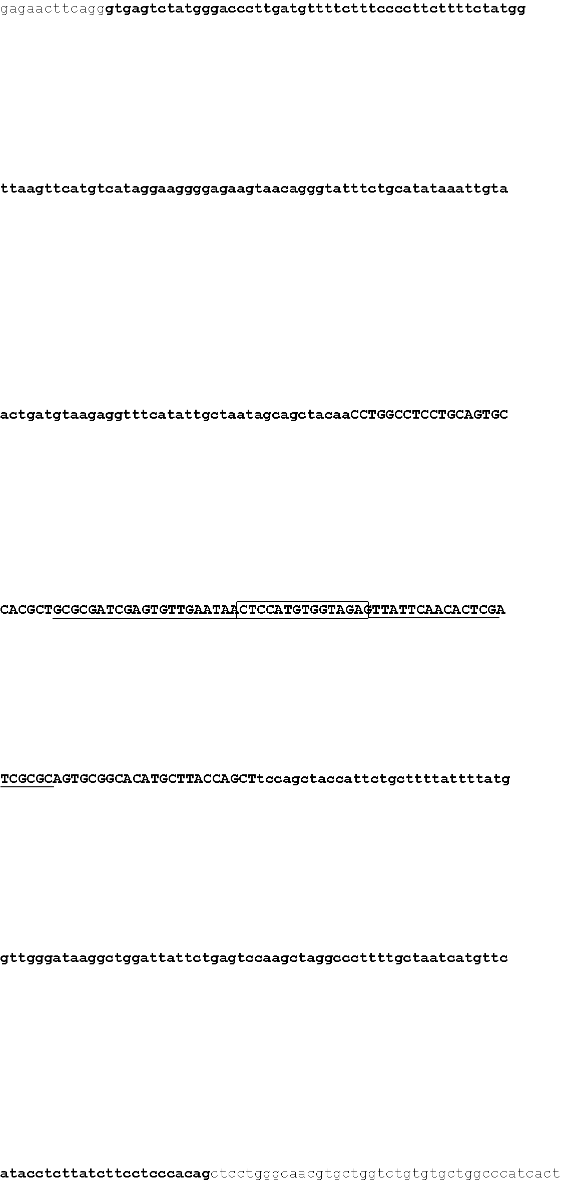

ttggcaaagaattcaccccaccagtgcaggctgcctatcagaaagtggtggctggtgtggctaatgccc
tggcccacaagtatcactaagctcgctttcttgctgtccaatttctattaaaggttcctttgttcccta
agtccaactactaaactgggggatattatgaagggccttgagcatctggattctgcctaataaaaaaca
tttattttcattgcaatgatgtatttaaattatttctgaatattttactaaaaagggaatgtgggaggt
cagtgcatttaaaacataaagaaatgaagagctagttcaaaccttgggaaaatacactatatcttaaac
tccatgaaagaaggtgaggctgcaaacagctaatgcacattggcaacagcccctgatgcctatgcctta
ttcatccctcagaaaaggattcaagtagaggcttgatttggaggttaaagttttgctatgctgtatttt
acattacttattgttttagctgtcctcatggtacgtaccgataaaattttgaattttgtaatttgtttt
tgtaattctttagtttgtatgtctgttgctattatgtctactattctttcccctgcactgtacccccca
atccccccttttcttttaaaagttaaccgataccgtcgagatccgttcactaatcgaatggatctgtct
ctgtctctctctccaccttcttcttctattccttcgggcctgtcgggtcccctcggggttgggaggtgg
gtctgaaacgataatggtgaatatccctgcctaactctattcactatagaaagtacagcaaaaactatt
cttaaacctaccaagcctcctactatcattatgaataattttatataccacagccaatttgttatgtta
aaccaattccacaaacttgcccatttatctaattccaataattcttgttcattcttttcttgctggttt
tgcgattcttcaattaaggagtgtattaagcttgtgtaattgttaatttctctgtcccactccatccag
gtcgtgtgattccaaatctgttccagagatttattactccaactagcattccaaggcacagcagtggtg
caaatgagttttccagagcaaccccaaatccccaggagctgttgatccttt

In some embodiments, the transgene of the present invention is under the transcriptional control of a promoter. As used herein, the terms “promoter” has its general meaning in the art and refers to a segment of a nucleic acid sequence, typically but not limited to DNA that controls the transcription of the nucleic acid sequence to which it is operatively linked. The promoter region includes specific sequences that are sufficient for RNA polymerase recognition, binding and transcription initiation. In addition, the promoter region can optionally include sequences which modulate this recognition, binding and transcription initiation activity of RNA polymerase. The skilled person will be aware that promoters are built from stretches of nucleic acid sequences and often comprise elements or functional units in those stretches of nucleic acid sequences, such as a transcription start site, a binding site for RNA polymerase, general transcription factor binding sites, such as a TATA box, specific transcription factor binding sites, and the like. Further regulatory sequences may be present as well, such as enhancers, and sometimes introns at the end of a promoter sequence.

As used herein, the terms “operably linked”, or “operatively linked” are used interchangeably herein, and refer to the functional relationship of the nucleic acid sequences with regulatory sequences of nucleotides, such as promoters, enhancers, transcriptional and translational stop sites, and other signal sequences and indicates that two or more DNA segments are joined together such that they function in concert for their intended purposes. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined.

In some embodiments, the transgene of the present invention is placed under the transcriptional control of the HBB promoter and key regulatory elements from the 16-kb human β-locus control region (βLCR), which is essential for high and regulated expression of the endogenous HBB gene family. In some embodiments, the key regulatory elements consists of the 2 DNase I hypersensitive sites HS2 and HS3.

In some embodiments, the transgene is operatively linked to further regulatory sequences. As used herein, the term “regulatory sequence” is used interchangeably with “regulatory element” herein and refers to a segment of nucleic acid, typically but not limited to DNA, that modulate the transcription of the nucleic acid sequence to which it is operatively linked, and thus acts as a transcriptional modulator. A regulatory sequence often comprises nucleic acid sequences that are transcription binding domains that are recognized by the nucleic acid-binding domains of transcriptional proteins and/or transcription factors, enhancers or repressors etc.

In some embodiments, the sequence of the transgenes is codon-optimized. As used herein, the term “codon-optimized” refers to nucleic sequence that has been optimized to increase expression by substituting one or more codons normally present in a coding sequence with a codon for the same (synonymous) amino acid. In this manner, the protein encoded by the gene is identical, but the underlying nucleobase sequence of the gene or corresponding mRNA is different. In some embodiments, the optimization substitutes one or more rare codons (that is, codons for tRNA that occur relatively infrequently in cells from a particular species) with synonymous codons that occur more frequently to improve the efficiency of translation. For example, in human codon-optimization one or more codons in a coding sequence are replaced by codons that occur more frequently in human cells for the same amino acid. Codon optimization can also increase gene expression through other mechanisms that can improve efficiency of transcription and/or translation. Strategies include, without limitation, increasing total GC content (that is, the percent of guanines and cytosines in the entire coding sequence), decreasing CpG content (that is, the number of CG or GC dinucleotides in the coding sequence), removing cryptic splice donor or acceptor sites, and/or adding or removing ribosomal entry sites, such as Kozak sequences. Desirably, a codon-optimized gene exhibits improved protein expression, for example, the protein encoded thereby is expressed at a detectably greater level in a cell compared with the level of expression of the protein provided by the wildtype gene in an otherwise similar cell.

In some embodiments, the transgene is inserted in a viral vector, and in particular in a retroviral vector. As used herein, the term “viral vector” refer to a virion or virus particle that functions as a nucleic acid delivery vehicle and which comprises a vector genome packaged within the virion or virus particle. As used herein, the term “retroviral vector” refers to a vector containing structural and functional genetic elements that are primarily derived from a retrovirus. In some embodiments, the retroviral vector of the present invention derives from a retrovirus selected from the group consisting of alpharetroviruses (e.g., avian leukosis virus), betaretroviruses (e.g., mouse mammary tumor virus), gammaretroviruses (e.g., murine leukemia virus), deltaretroviruses (e.g., bovine leukemia virus), epsilonretroviruses (e.g., Walley dermal sarcoma virus), lentiviruses (e.g., HIV-1, HIV-2) and spumaviruses (e.g., human spumavirus). In some embodiments, the retroviral vector of the present invention is a replication deficient retroviral virus particle, which can transfer a foreign imported RNA of a gene instead of the retroviral mRNA.

In some embodiments, the retroviral vector of the present invention is a lentiviral vector. As used herein, the term “lentiviral vector” refers to a vector containing structural and functional genetic elements that are primarily derived from a lentivirus. In some embodiments, the lentiviral vector of the present invention is selected from the group consisting of HIV-1, HIV-2, SIV, FIV, EIAV, BIV, VISNA and CAEV vectors. In some embodiments, the lentiviral vector is a HIV-1 vector. The structure and composition of the vector genome used to prepare the retroviral vectors of the present invention are in accordance with those described in the art. Especially, minimum retroviral gene delivery vectors can be prepared from a vector genome, which only contains, apart from the nucleic acid molecule of the present invention, the sequences of the retroviral genome which are non-coding regions of said genome, necessary to provide recognition signals for DNA or RNA synthesis and processing. In some embodiment, the retroviral vector genome comprises all the elements necessary for the nucleic import and the correct expression of the polynucleotide of interest (i.e. the transgene). As examples of elements that can be inserted in the retroviral genome of the retroviral vector of the present invention are at least one (preferably two) long terminal repeats (LTR), such as a LTR5′ and a LTR3′, a psi sequence involved in the retroviral genome encapsidation, and optionally at least one DNA flap comprising a cPPT and a CTS domains. In some embodiments of the present invention, the LTR, preferably the LTR3′, is deleted for the promoter and the enhancer of U3 and is replaced by a minimal promoter allowing transcription during vector production while an internal promoter is added to allow expression of the transgene. In particular, the vector is a Self-INactivating (SIN) vector that contains a non-functional or modified 3′ Long Terminal Repeat (LTR) sequence. This sequence is copied to the 5′ end of the vector genome during integration, resulting in the inactivation of promoter activity by both LTRs. Hence, a vector genome may be a replacement vector in which all the viral coding sequences between the 2 long terminal repeats (LTRs) have been replaced by the nucleic acid molecule of the present invention.

In some embodiments, the retroviral vector genome is devoid of functional gag, pol and/or env retroviral genes. By “functional” it is meant a gene that is correctly transcribed, and/or correctly expressed. Thus, the retroviral vector genome of the present invention in this embodiment contains at least one of the gag, pol and env genes that is either not transcribed or incompletely transcribed; the expression “incompletely transcribed” refers to the alteration in the transcripts gag, gag-pro or gag-pro-pol, one of these or several of these being not transcribed. In some embodiments, the retroviral genome is devoid of gag, pol and/or env retroviral genes.

In some embodiments the retroviral vector genome is also devoid of the coding sequences for Vif-, Vpr-, Vpu- and Nef-accessory genes (for HIV-1 retroviral vectors), or of their complete or functional genes.

In some embodiments, the vector of the present invention comprises a packaging signal. A “packaging signal,” “packaging sequence,” or “psi sequence” is any nucleic acid sequence sufficient to direct packaging of a nucleic acid whose sequence comprises the packaging signal into a retroviral particle. The term includes naturally occurring packaging sequences and also engineered variants thereof. Packaging signals of a number of different retroviruses, including lentiviruses, are known in the art.

In some embodiments, the vector of the present invention comprises a Rev Response Element (RRE) to enhance nuclear export of unspliced RNA. RREs are well known to those of skill in the art. Illustrative RREs include, but are not limited to RREs such as that located at positions 7622-8459 in the HIV NL4-3 genome (Genbank accession number AF003887) as well as RREs from other strains of HIV or other retroviruses.

Typically, the retroviral vector of the present invention is non replicative i.e., the vector and retroviral vector genome are not able to form new particles budding from the infected host cell. This may be achieved by the absence in the retroviral genome of the gag, pol or env genes, as indicated in the above paragraph; this can also be achieved by deleting other viral coding sequence(s) and/or cis-acting genetic elements needed for particles formation.

The retroviral vectors of the present invention can be produced by any well-known method in the art including transient transfection (s) in cell lines. Use of stable cell lines may also be preferred for the production of the vectors. For instance, the retroviral vector of the present invention is obtainable by a transcomplementation system (vector/packaging system) by transfecting in vitro a permissive cell (such as 293T cells) with a plasmid containing the retroviral vector genome of the present invention, and at least one other plasmid providing, in trans, the gag, pol and env sequences encoding the polypeptides GAG, POL and the envelope protein(s), or for a portion of these polypeptides sufficient to enable formation of retroviral particles. As an example, permissive cells are transfected with a) transcomplementation plasmid, lacking packaging signal psi and the plasmid is optionally deleted of accessory genes vif, nef, vpu and/or vpr, b) a second plasmid (envelope expression plasmid or pseudotyping env plasmid) comprising a gene encoding an envelope protein(s) and c) a transfer vector plasmid comprising a recombinant retroviral genome, optionally carrying the deletion of the U3 promoter/enhancer region of the 3′ LTR, including, between the 5 ′and 3′ retroviral LTR sequences, a psi encapsidation sequence, a nuclear export element (preferably RRE element of HIV or other retroviruses equivalent), and the nucleic acid molecule of the present invention, and optionally a promoter and/or a sequences involved in the nuclear import (cPPT and CTS) of the RNA. Advantageously, the three plasmids used do not contain homologous sequence sufficient for recombination. Nucleic acids encoding gag, pol and env cDNA can be advantageously prepared according to conventional techniques, from viral gene sequences available in the prior art and databases. The trans-complementation plasmid provides a nucleic acid encoding the proteins retroviral gag and pol. These proteins are derived from a lentivirus, and most preferably, from HIV-1. The plasmid is devoid of encapsidation sequence, sequence coding for an envelope, accessory genes, and advantageously also lacks retroviral LTRs. Therefore, the sequences coding for gag and pol proteins are advantageously placed under the control of a heterologous promoter, e.g. cellular, viral, etc., which can be constitutive or regulated, weak or strong. It is preferably a plasmid containing the transcomplementing sequence Δpsi-CMV-gag-pol-PolyA. This plasmid allows the expression of all the proteins necessary for the formation of empty virions, except the envelope glycoproteins. The transcomplementation plasmid may advantageously comprise the TAT and REV genes. The transcomplementation plasmid is advantageously devoid of vif, vpr, vpu and/or nef accessory genes. It is understood that the gag and pol genes and genes TAT and REV can also be carried by different plasmids, possibly separated. In this case, several transcomplementation plasmids are used, each encoding one or more of said proteins. The promoters used in the transcomplementation plasmid, the envelope plasmid and the transfer vector plasmid respectively to promote the expression of gag and pol, of the coat protein, and the mRNA of the vector genome (including the transgene) are promoters identical or different, chosen advantageously from ubiquitous promoters or cell-specific, for example, the viral CMV, TK, RSV LTR promoters and the RNA polymerase III promoters such as U6 or H1. For the production of the retroviral vector of the present invention, the plasmids described above can be introduced into appropriate cells and viruses produced are harvested. The cells used may be any cell particularly eukaryotic cells, in particular mammalian, e.g. human or animal. They can be somatic or embryonic stem or differentiated cells. Typically the cells include 293T cells, fibroblast cells, hepatocytes, muscle cells (skeletal, cardiac, smooth, blood vessel, etc.), nerve cells (neurons, glial cells, astrocytes) of epithelial cells, renal, ocular etc. It may also include, insect, plant cells, yeast, or prokaryotic cells. It can also be cells transformed by the SV40 T antigen. The genes gag, pol and env encoded in plasmids can be introduced into cells by any method known in the art, suitable for the cell type considered. Usually, the cells and the plasmids are contacted in a suitable device (plate, dish, tube, pouch, etc. . . . ), for a period of time sufficient to allow the transfer of the plasmid in the cells. Typically, the plasmid is introduced into the cells by calcium phosphate precipitation, electroporation, or by using one of transfection-facilitating compounds, such as lipids, polymers, liposomes and peptides, etc. The calcium phosphate precipitation is preferred. The cells are cultured in any suitable medium such as RPMI, DMEM, a specific medium devoid of fetal calf serum, etc. After transfection, the retroviral vectors of the present invention may be purified from the supernatant of the cells. Purification of the retroviral vector to enhance the concentration can be accomplished by any suitable method, such as by chromatography techniques (e.g., column or batch chromatography).

The vector of the present invention is particularly suitable for driving the targeted expression of the transgene in a host cell. Accordingly, a further object of the present invention relates to a method of obtaining a population of host cells transduced with the transgene of the present invention, which comprises the step of transducing a population of host cells in vitro or ex vivo with the vector of the present invention.

The term “transduction” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transduced”.

In some embodiments, the host cell is selected from the group consisting of hematopoietic stem/progenitor cells, hematopoietic progenitor cells, hematopoietic stem cells (HSCs), pluripotent cells (i.e. embryonic stem cells (ES) and induced pluripotent stem cells (iPS)).

Typically, the host cell results from a stem cell mobilization. As used herein, the term “mobilization” or “stem cell mobilization” refers to a process involving the recruitment of stem cells from their tissue or organ of residence to peripheral blood following treatment with a mobilization agent. This process mimics the enhancement of the physiological release of stem cells from tissues or organs in response to stress signals during injury and inflammation. The mechanism of the mobilization process depends on the type of mobilization agent administered. Some mobilization agents act as agonists or antagonists that prevent the attachment of stem cells to cells or tissues of their microenvironment. Other mobilization agents induce the release of proteases that cleave the adhesion molecules or support structures between stem cells and their sites of attachment. As used herein, the term “mobilization agent” refers to a wide range of molecules that act to enhance the mobilization of stem cells from their tissue or organ of residence, e.g., bone marrow (e.g., CD34+ stem cells) and spleen (e.g., Hox11+ stem cells), into peripheral blood. Mobilization agents include chemotherapeutic drugs, e.g., cyclophosphamide and cisplatin; cytokines, and chemokines, e.g., granulocyte colony-stimulating factor (G-CSF), granulocyte-macrophage colony-stimulating factor (GM-CSF), stem cell factor (SCF), Fms-related tyrosine kinase 3 (flt-3) ligand, stromal cell-derived factor 1 (SDF-1); agonists of the chemokine (C-C motif) receptor 1 (CCR1), such as chemokine (C-C motif) ligand 3 (CCL3, also known as macrophage inflammatory protein-1α (Mip-1α)); agonists of the chemokine (C—X-C motif) receptor 1 (CXCR1) and 2 (CXCR2), such as chemokine (C—X-C motif) ligand 2 (CXCL2) (also known as growth-related oncogene protein-β (Gro-β)), and CXCL8 (also known as interleukin-8 (IL-8)); agonists of CXCR4, such as CTCE-02142, and Met-SDF-1,; Very Late Antigen (VLA)-4 inhibitors; antagonists of CXCR4, such as TG-0054, plerixafor (also known as AMD3100), and AMD3465, or any combination of the previous agents. A mobilization agent increases the number of stem cells in peripheral blood, thus allowing for a more accessible source of stem cells for use in transplantation, organ repair or regeneration, or treatment of disease.

As used herein, the term “hematopoietic stem cell” or “HSC” refers to blood cells that have the capacity to self-renew and to differentiate into precursors of blood cells. These precursor cells are immature blood cells that cannot self-renew and must differentiate into mature blood cells. Hematopoietic stem progenitor cells display a number of phenotypes, such as Lin-CD34+CD38-CD90+CD45RA-, Lin-CD34+CD38-CD90-CD45RA-, Lin-CD34+CD38+IL-3aloCD45RA-, and Lin-CD34+CD38+CD10+(Daley et al., Focus 18:62-67, 1996; Pimentel, E., Ed., Handbook of Growth Factors Vol. III: Hematopoietic Growth Factors and Cytokines, pp. 1-2, CRC Press, Boca Raton, Fla., 1994). Within the bone marrow microenvironment, the stem cells self-renew and maintain continuous production of hematopoietic stem cells that give rise to all mature blood cells throughout life. In some embodiments, the hematopoietic progenitor cells or hematopoietic stem cells are isolated form peripheral blood cells.

As used herein, the term “peripheral blood cells” refer to the cellular components of blood, including red blood cells, white blood cells, and platelets, which are found within the circulating pool of blood. In some embodiments, the host cell is a bone marrow derived stem cell.

As used herein the term “bone marrow-derived stem cells” refers to stem cells found in the bone marrow. Stem cells may reside in the bone marrow, either as an adherent stromal cell type that possess pluripotent capabilities, or as cells that express CD34 or CD45 cell-surface protein, which identifies hematopoietic stem cells able to differentiate into blood cells.

Typically, the host cell is isolated. As used herein, the term “isolated cell” refers to a cell that has been removed from an organism in which it was originally found, or a descendant of such a cell. Optionally the host cell has been cultured in vitro, e.g., in the presence of other cells. Optionally the host cell is later introduced into a second organism or reintroduced into the organism from which it (or the cell from which it is descended) was isolated. As used herein, the term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched.

Methods for transducing host cells are well known in the art. In some embodiments, the host cells may be cultured in the presence of the retroviral vector for a duration of about 10 minutes to about 72 hours, about 30 minutes to about 72 hours, about 30 minutes to about 48 hours, about 30 minutes to about 24 hours, about 30 minutes to about 12 hours, about 30 minutes to about 8 hours, about 30 minutes to about 6 hours, about 30 minutes to about 4 hours, about 30 minutes to about 2 hours, about 1 hour to about 2 hours, or any intervening period of time. During transduction, the host cells may be cultured in media suitable for the maintenance, growth, or proliferation of the host cells. Suitable culture media and conditions are well known in the art. Such media include, but are not limited to, Dulbecco's Modified Eagle's Medium® (DMEM), DMEM F12 Medium®, Eagle's Minimum Essential Medium®, F-12K Medium®, Iscove's Modified Dulbecco's Medium®, RPMI-1640 Medium®, and serum-free medium for culture and expansion of hematopoietic cells SFEM®. Many media are also available as low-glucose formulations, with or without sodium pyruvate. During transduction, the host cells may be cultured under conditions that promote the expansion of stem cells or progenitor cells. Any method known in the art may be used. In some embodiments, during transduction, the host cells are cultured in the presence of one or more growth factors that promote the expansion of stem cells or progenitor cells. Examples of growth factors that promote the expansion of stem cells or progenitor cells include, but are not limited to, fetal liver tyrosine kinase (Flt3) ligand, stem cell factor (SCF), and interleukins 6 and 11, which have been demonstrated to promote self-renewal of murine hematopoietic stem cells. Others include Sonic hedgehog, which induces the proliferation of primitive hematopoietic progenitors by activation of bone morphogenetic protein 4, Wnt3a, which stimulates self-renewal of HSCs, brain derived neurotrophic factor (BDNF), epidermal growth factor (EGF), fibroblast growth factor (FGF), ciliary neurotrophic factor (CNF), transforming growth factor-β (TGF-β), a fibroblast growth factor (FGF, e.g., basic FGF, acidic FGF, FGF-17, FGF-4, FGF-5, FGF-6, FGF-8b, FGF-8c, FGF-9), granulocyte colony stimulating factor (GCSF), a platelet derived growth factor (PDGF, e.g., PDGFAA, PDGFAB, PDGFBB), granulocyte macrophage colony stimulating factor (GMCSF), stromal cell derived factor (SCDF), insulin like growth factor (IGF), thrombopoietin (TPO) or interleukin-3 (IL-3). In some embodiments, before transduction, the host cells are cultured in the presence of one or more growth factors that promote expansion of stem cells or progenitor cells. In some embodiments, transduction efficiency is significantly increased by contacting the host cells with the retroviral vector in presence of one or more compounds that stimulate the prostaglandin EP receptor signaling pathway, selected from the group consisting of: a prostaglandin, PGE2; PGD2; PGI2; Linoleic Acid; 13(s)-HODE; LY171883; Mead Acid; Eicosatrienoic Acid; Epoxyeicosatrienoic Acid; ONO-259; Cay1039; a PGE2 receptor agonist; 16,16-dimethyl PGE2; 19(R)-hydroxy PGE2; 16,16-dimethyl PGE2 p-(p-acetamidobenzamido) phenyl ester; 11-deoxy-16,16-dimethyl PGE2; 9-deoxy-9-methylene-16,16-dimethyl PGE2; 9-deoxy-9-methylene PGE2; Butaprost; Sulprostone; PGE2 serinol amide; PGE2 methyl ester; 16-phenyl tetranor PGE2; 15(S)-15-methyl PGE2; 15(R)-15-methyl PGE2; BIO; 8-bromo-cAMP; Forskolin; Bapta-AM; Fendiline; Nicardipine; Nifedipine; Pimozide; Strophanthidin; Lanatoside; L-Arg; Sodium Nitroprusside; Sodium Vanadate; Bradykinin; Mebeverine; Flurandrenolide; Atenolol; Pindolol; Gaboxadol; Kynurenic Acid; Hydralazine; Thiabendazole; Bicuclline; Vesamicol; Peruvoside; Imipramine; Chlorpropamide; 1,5-Pentamethylenetetrazole; 4-Aminopyridine; Diazoxide; Benfotiamine; 12-Methoxydodecenoic acid; N-Formyl-Met-Leu-Phe; Gallamine; IAA 94; and Chlorotrianisene.

Typically, the host cells can be then delivered to a subject in which the transgene encoding for the anti-sickling β-globin will be expressed concomitantly with the artificial miRNA of the present invention that will thus allow the re-expression of gamma globin (that is repressed by BCL11A).

As used herein, the term “gamma globin” or “γ-globin” has its general meaning in the art and refers to protein that is encoded in human by the HBG1 and HBG2 genes.

Thus the host cells of the present invention will express a suitable amount of the anti-sickling β-globin and a suitable amount of γ-globin and thus can particularly useful for the treatment of hemoglobinopathies.

Accordingly, a further object of the present invention relates to a method of treating a hemoglobinopathy in a subject in need thereof, the method comprising transplanting a therapeutically effective amount of a population of host cells obtained by the method as above described.

In some embodiments, the population of host cells is autologous to the subject, meaning the population of cells is derived from the same subject.

As used herein, the term “hemoglobinopathy” has its general meaning in the art and refers to any defect in the structure or function of any hemoglobin of an individual, and includes defects in the primary, secondary, tertiary or quaternary structure of hemoglobin caused by any mutation, such as deletion mutations or substitution mutations in the coding regions of the HBB gene, or mutations in, or deletions of, the promoters or enhancers of such gene that cause a reduction in the amount of hemoglobin produced as compared to a normal or standard condition. In some embodiments, the hemoglobinopathy is a β-hemoglobinopathy. In some embodiments, the β-hemoglobinopathy is a sickle cell disease. As used herein, “sickle cell disease” has its general meaning in the art and refers to a group of autosomal recessive genetic blood disorders, which results from mutations in a globin gene and which is characterized by red blood cells that assume an abnormal, rigid, sickle shape. They are defined by the presence of βS-globin gene coding for a β-globin chain variant in which glutamic acid is substituted by valine at amino acid position 6 of the peptide: incorporation of the βS-globin in the Hb tetramers (HbS, sickle Hb) leads to Hb polymerization and to a clinical phenotype. The term includes sickle cell anemia (HbSS), sickle-hemoglobin C disease (HbSC), sickle beta-plus-thalassaemia (HbS/β+), or sickle beta-zerothalassaemia (HbS/β0). In some embodiments, the hemoglobinopathy is a β-thalassemia. As used herein, the term “β-thalassemia” refers to a hemoglobinopathy that results from an altered ratio of α-globin to β-like globin polypeptide chains resulting in the underproduction of normal hemoglobin tetrameric proteins and the precipitation of free, unpaired α-globin chains.

By a “therapeutically effective amount” is meant a sufficient amount of population of host cells to treat the disease at a reasonable benefit/risk ratio applicable to any medical treatment. It will be understood that the total usage compositions of the present invention will be decided by the attending physician within the scope of sound medical judgment. The specific therapeutically effective dose level for any particular patient will depend upon a variety of factors including the age, body weight, general health, sex and diet of the patient, the time of administration, route of administration, the duration of the treatment, drugs used in combination or coincidental with the population of cells, and like factors well known in the medical arts. In some embodiments, the host cells are formulated by first harvesting them from their culture medium, and then washing and concentrating the host cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the relative representation of the host cells with the desired specificity, on the age and weight of the recipient, and on the severity of the targeted condition. This amount of cells can be as low as approximately 10³/kg, preferably 5×10³/kg; and as high as 10⁷/kg, preferably 10⁸/kg. The number of cells will depend upon the ultimate use for which the composition is intended, as will the type of cells included therein. Typically, the minimal dose is 2 million of cells per kg. Usually 2 to 20 million of cells are injected in the subject. The desired purity can be achieved by introducing a sorting step. For uses provided herein, the host cells are generally in a volume of a liter or less, can be 500 ml or less, even 250 ml or 100 ml or less. The clinically relevant number of cells can be apportioned into multiple infusions that cumulatively equal or exceed the desired total amount of cells.

The invention will be further illustrated by the following figures and examples. However, these examples and figures should not be interpreted in any way as limiting the scope of the present invention.

### FIGURES

FIG. 1: Introduction of the modified shRNA #5 embedded in the miR-223 backbone in intron 2 of the βAS3 transgene. (A) The amiR is composed by a shRNA embedded in the miR-223 backbone (top panel). The sequence of the different amiR components is shown (bottom panel-(SEQ ID NO: 27)). (B) The shRNA #5 embedded in the miR-223 backbone (SEQ ID NO: 28). This amiR targets BCLL11A-XL RNA. (C) The amiR was inserted inside intron 2 of the βAS3 transgene between positions 85 and 86 (where a 593-bp region was deleted) or between positions 146 and 147 (βAS3-miR/int2_del and βAS3-miR/int2, respectively).

FIG. 2: The presence of the amiR does not affect gene transfer efficiency in HUDEP-2 cells. VCN/cell was measured by ddPCR in HUDEP-2 cells transduced with αAS3, βAS3-miR/int2_del or βAS3-miR/int2 LVs at MOI 1, 5, 10 and 15. After transduction, cells were grown for 14 days before measuring the VCN/cell.

FIG. 3: The amiR reduces BCL11A XL mRNA expression levels. BCL11A XL mRNA levels were measured by RT-qPCR in mock- and LV-transduced HUDEP-2 cells after 9 days of differentiation. mRNA levels were normalized to LMNB2 expression.

FIG. 4: βAS3 transgene expression is not affected by the insertion of the amiR in intron 2. βAS3 mRNA levels were measured by RT-qPCR in mock- and LV-transduced HUDEP-2 cells after 9 days of differentiation. βAS3 mRNA levels were normalized to HBA expression. We plotted βAS3 mRNA levels per VCN. No significant statistical difference was observed between the 3 LVs.

FIG. 5: Induction of HBG1 and 2 gene expression upon BCL11A-XL silencing. HBG1/2 mRNAs were measured by RT-qPCR in HUDEP-2 cells after 9 days of differentiation. HBG1/2 mRNA levels were normalized to HBA expression. We plotted HBG1/2 mRNA levels per VCN. No significant difference was observed between the AS3-miR/int2_del and βAS3-miR/int23 LVs. HBG1/2 mRNA levels were significantly higher in βAS3-miR/int2_del- and βAS3-miR/int23-transduced cells than in βAS3-transduced samples (One-way ANOVA test; *** P<0.001).

FIG. 6: HbF induction upon BCL11A-XL silencing. (A) Representative flow cytometry analysis of HbF expression in terminally differentiated CD235a^{high} HUDEP-2 cells after 9 days of differentiation. (B) Graphs showing the percentage of HbF⁺ cells and the corresponding mean fluorescence intensities (MFI). (C) Graphs showing the β-like-globin/α-ratios, as determined by reverse-phase HPLC.

FIG. 7: Erythroid differentiation is not altered upon transduction of HD HSPCs with the BCL11A amiR-expressing LVs. Flow cytometry analysis of CD71 (A), CD36 (B) and CD235a (C) expression. We plotted the percentage of erythroid cells derived from HD CD34⁺ HSPCs expressing CD71, CD36 or CD235a. These erythroid surface markers were analyzed along the differentiation at day 6 (D6), day 13 (D13), day 16 (D16), and day 20 (D20). The expression of the early erythroid markers CD36 and CD71 decreased along the differentiation while the expression of the late erythroid marker CD235a increased. Erythroid differentiation was not impacted in samples transduced with the LVs containing the amiR BCL11A (βAS3-miR/int2 and βAS3-miR/int2_del) compared to control cells (mock-transduced cells (Mock), cells transduced with the LV containing either the βAS3 alone (βAS3), or the βAS3 and a non-targeting (nt) amiR (βAS3-miR #nt/int2 and βAS3-miR #nt/int2_del).

FIG. 8: Transduction of HD HSPCs with BCL11A amiR-expressing LVs does not impact the enucleation rate of RBCs derived from HD CD34⁺ HSPCs. (A, B). Flow cytometry analysis of DRAQ5⁺ nucleated and DRAQ5⁻ enucleated RBCs-derived HD CD34⁺ HSPCs. We measured the percentage of enucleated RBCs along the differentiation at day 6 (D6), day 13 (D13), day 16 (D16) and day 20 (D20). Enucleated RBCs were detected from day 13 and their proportion increased to up to 90% at D20. Enucleation was not impacted in samples transduced with the LVs containing the amiR BCL11A (βAS3-miR/int2 and βAS3-miR/int2_del) compared to control cells (mock-transduced cells (Mock), cells transduced with the LV containing either the βAS3 alone (βAS3), or the βAS3 and a non-targeting (nt) amiR (βAS3-miR #nt/int2 and βAS3-miR #nt/int2_del)).

FIG. 9: HBG genes are de-repressed in primary erythroid cells transduced with the BCL11A amiR-expressing LVs. HBG1 and HBG2 mRNA levels were measured by RT-qPCR in erythroid precursors derived from HD CD34⁺ HSPCs after 13 days of differentiation. HBG mRNA levels were normalized to HBA gene expression. We plotted HBG mRNA levels per VCN. HBG mRNA levels were higher in transduced cells with LVs containing the BCL11A amiR (βAS3-miR/int2 and βAS3-miR/int2_del) than in control cells transduced with LV containing the βAS3 alone (βAS3) or the βAS3 and a non-targeting (nt) amiR (βAS3-miR #nt/int2 and βAS3-miR #nt/int2_del).

FIG. 10: γ-globin induction in primary erythroid cells transduced with the BCL11A amiR-expressing LVs. (A) Western blot analysis of γ-globin expression in RBCs derived from HD CD34⁺ HSPCs after 16 days of differentiation. α-globin was used as the loading control. γ-globin expression was normalized to α-globin. (B) We plotted γ-globin chain expression levels per VCN and γ-globin chain fold-increase between control (βAS3-miR #nt) and BCL11A-miR transduced cells (βAS3-miR) for the LVs containing the BCL11A amiR in position int2 or int2_del.

γ-globin chain levels were higher in BCL11A amiR-transduced cells (βAS3-miR/int2 and βAS3-miR/int2_del) compared to control cells transduced with LV containing the βAS3 alone (βAS3) or the βAS3 and a non-targeting (nt) amiR (βAS3-miR #nt/int2 and βAS3-miR #nt/int2_del).

FIG. 11: Increased therapeutic globin levels in cells transduced with BCL11A amiR-expressing LVs. Graphs showing the β-like globin/α-globin ratios (A) and the (βAS3+γ)/VCN ratios (B) as measured by RP-HPLC and the percentage of hemoglobin tetramers (C) and the (HbF+HbAS3)/VCN ratios (D) as determined by cation exchange-HPLC (CE-HPLC). In graphs A and C, the VCN is indicated.

Globin chain and hemoglobin expression was assessed in RBCs derived from HD CD34⁺ HSPCs after 16 days of differentiation. γ-globin and HbF expression were higher in BCL11A amiR-transduced cells (βAS3-miR/int2 and βAS3-miR/int2_del) compared to mock-transduced cells (Mock) or cells transduced with LV expressing βAS3 and a non-targeting (nt) amiR (βAS3-miR #nt/int2). γ-globin de-repression coupled with βAS3 transgene expression leads to a 2-fold increase in therapeutic globins (βAS3+γ) and hemoglobin tetramers (HbF+HbAS3) per VCN. Fold-increase is indicated above the graphs.

### Example: A Novel Lentiviral Vector for Gene Therapy of B-Hemoglobinopathies: Co-Expression of a Potent Anti-Sickling Transgene and a MicroRNA Downregulating BCL11A

Methods:

Lentiviral Vector Production and Titration

Third-generation LVs were produced by calcium phosphate transient transfection of HEK293T cells with the transfer vector (pCCL.βAS3, pCCL.βAS3-miR/int2_del or βAS3-miR/int2, pCCL.βAS3-miR #nt/int2_del or βAS3-miR #nt/int2), the packaging plasmid pHDMH gpm2 (encoding gag/pol), the Rev-encoding plasmid pBA Rev, and the vesicular stomatitis virus glycoprotein G (VSV-G) envelope-encoding plasmid pHDM-G. The viral infectious titer, expressed as transduction units per ml (TU/ml) was measured in HCT116 cells after transduction using serial vector dilutions. Three days after transduction, genomic DNA was extracted and the vector copy number (VCN) per cell was measured by qPCR. The VCN per cell was used to calculate the viral infectious titer.

HUDEP-2 Cell Culture, Differentiation and Transduction

HUDEP-2 cells (HUDEP-2) were cultured and differentiated as previously described (Antoniani et al., 2018; Canver et al., 2015; Kurita et al., 2013). HUDEP-2 cells were expanded in a basal medium composed of StemSpan SFEM (Stem Cell Technologies) supplemented with 10⁻⁶M dexamethasone (Sigma), 100 ng/ml human stem cell factor (hSCF) (Peprotech), 3 IU/ml erythropoietin (EPO) Eprex (Janssen-Cilag, France), 100 U/ml L-glutamine (Life Technologies), 2 mM penicillin/streptomycin and 1 μg/ml doxycycline (Sigma). HUDEP-2 cells were transduced at a cell concentration of 10⁶ cells/ml in basal medium supplemented with 4 ug/ml protamine sulfate (Choay). After 24 h, cells were washed and cultured in fresh basal medium. Cells were differentiated for 9 days in Iscove's Modified Dulbecco's Medium (IMDM) (Life Technologies) supplemented with 330 μg/ml holo-transferrin (Sigma), 10 μg/ml recombinant human insulin (Sigma), 2 IU/ml heparin (Sigma), 5% human AB serum (Eurobio AbCys), 3 IU/mL erythropoietin, 100 ng/mL human SCF, 1 μg/ml doxycycline, 100 U/ml L-glutamine, and 2 mM penicillin/streptomycin.

HSPC Purification and Transduction

Human adult HSPCs were obtained from healthy donors (HD). Written informed consent was obtained from all subjects. All experiments were performed in accordance with the Declaration of Helsinki. The study was approved by the regional investigational review board (reference, DC 2014-2272, CPP Ile-de-France II “Hôpital Necker-Enfants malades”). HSPCs were purified by immunomagnetic selection (Miltenyi Biotec) after immunostaining using the CD34 MicroBead Kit (Miltenyi Biotec).

CD34⁺ cells were thawed and cultured for 24 h at a concentration of 10⁶ cells/mL in pre-activation medium composed of X-VIVO 20 supplemented with penicillin/streptomycin (Gibco) and recombinant human cytokines: 300 ng/mL SCF, 300 ng/mL Flt-3 L, 100 ng/mL TPO, 20 ng/mL interleukin-3 (IL-3) (Peprotech) and 10 mM SR1 (StemCell). After pre-activation, cells (3.10⁶ cells/mL) were cultured in pre-activation medium supplemented with 10 μM PGE2 (Cayman Chemical) on RetroNectin coated plates (10 μg/cm2, Takara Bio) for at least 2 h. Cells (3.10⁶ cells/mL) were then transduced for 24 h on RetroNectin coated plates in the pre-activation medium supplemented with 10 μM PGE2, protamine sulfate (4 μg/mL, Protamine Choay) and Lentiboost (1 mg/ml, SirionBiotech).

In Vitro Erythroid Differentiation

Mature RBCs from mock- and LV-transduced CD34⁺ HSPCs were generated using a three-step protocol (Weber et al., 2018). Briefly, from day 0 to 6, cells were grown in a basal erythroid medium (BEM) supplemented with SCF, IL3, erythropoietin (EPO) (Eprex, Janssen-Cilag) and hydrocortisone (Sigma). From day 6 to 20, they were cultured on a layer of murine stromal MS-5 cells in BEM supplemented with EPO from day 6 to day 9 and without cytokines from day 9 to day 20. From day 13 to 20, human AB serum was added to the BEM.

Vector Copy Number Quantification by ddPCR

Genomic DNA was extracted from HUDEP-2 cells 14 days after transduction or from primary erythroid cells at day 13 of differentiation using the PureLink Genomic DNA Mini Kit (Invitrogen). DNA was digested using Dral restriction enzyme (NEB) at 37° C. for 30 min and then mixed with the ddPCR reaction mix composed of 2X ddPCR SuperMix for probes (no dUTP) (Bio-Rad), forward (for) and reverse (rev) primers (at a final concentration of 900 nM) and probes (at a final concentration of 250 nM). We used probes and primers specific for: (i) albumin (VIC-labeled ALB probe with a QSY quencher, 5′-CCTGTCATGCCCACACAAATCTCTCC-3′ (SEQ ID NO: 11); FOR ALB primer, 5′-GCTGTCATCTCTTGTGGGCTGT-3′(SEQ ID NO: 12); REV ALB primer, 5′ ACTCATGGGAGCTGCTGGTTC-3′ (SEQ ID NO: 13)), and for (ii) the LV (FAM-labeled LV probe with a MGB quencher, 5′-CGCACGGCAAGAGGCGAGG-3′ (SEQ ID NO: 14); FOR LV primer 5′-TCCCCCGCTTAATACTGACG-3′(SEQ ID NO: 15); REV LV primer 5′-CAGGACTCGGCTTGCTGAAG-3′ (SEQ ID NO: 16)). Droplets were generated using a QX200 droplet generator (Bio-Rad) with droplet generation oil for probes (Bio-Rad) onto a DG8 cartridge (Bio-Rad) and transferred on a semi-skirted 96 well plate (Eppendorf AG). After sealing with a pierce-able foil heat seal using a PX1 PCR plate sealer (Bio-Rad), the plate was loaded on a SimpliAmp Thermal Cycler (ThermoFisher Scientific) for PCR amplification using the following conditions: 95° C. for 10 min, followed by 40 cycles at 94° C. for 30 sec and 60° C. for 1 min, and by a final step at 98° C. for 10 min. The plate was analyzed using the QX200 droplet reader (Bio-Rad) (channel 1: FAM, channel 2: VIC) and analyzed using the QuantaSoft analysis software (Bio-Rad), which quantifies positive and negative droplets and calculate the starting DNA concentration using a Poisson algorithm. The VCN) per cell were calculated as (LV copies*2)/(albumin copies).

RT-qPCR Analysis

RNA was extracted from HUDEP-2 cells after 9 days of differentiation or from primary erythroid cells at day 13 of differentiation using the RNeasy micro kit (QIAGEN). Reverse transcription of mRNA was performed using the SuperScript III First-Strand Synthesis System for RT-PCR (Invitrogen) with oligo(dT)₂₀ primers. qPCR was performed using the SYBR green detection system (BioRad). We used the following primers: βAS3 FOR, 5′-GCCACCACTTTCTGATAGGCAG-3′ (SEQ ID NO: 17); βAS3 REV, 5′-AAGGGCACCTTTGCCCAG-3′ (SEQ ID NO: 18); BCL11A-XL FOR, 5′-ATGCGAGCTGTGCAACTATG-3′ (SEQ ID NO: 19); BCL11A-XL REV, 5′-GTAAACGTCCTTCCCCACCT-3′ (SEQ ID NO: 20); HBG1/2 FOR, 5′ CCTGTCCTCTGCCTCTGCC-3′ (SEQ ID NO: 21); HBG1/2 REV, 5′-GGATTGCCAAAACGGTCAC-3′ (SEQ ID NO: 22); LMNB2 FOR, 5′-AGTTCACGCCCAAGTACATC-3′ (SEQ ID NO: 23); LMNB2 REV, 5′-CTTCACAGTCCTCATGGCC-3′(SEQ ID NO: 24); HBA FOR, 5′-CGGTCAACTTCAAGCTCCTAA-3′(SEQ ID NO: 25); HBA REV, 5′-ACAGAAGCCAGGAACTTGTC-3′(SEQ ID NO: 26). The samples were analyzed with the ViiA 7 Real-Time PCR System and software (Applied Biosystems).

Flow Cytometry

After nine days of differentiation, HUDEP-2 cells were stained with a monoclonal mouse anti-human CD235a antibody (clone GA-R2, BD Biosciences), then fixed and permeabilized with the fixation/permeabilization solution kit (BD Biosciences) and stained with a monoclonal mouse anti-human HbF antibody (clone HBF-1, ThermoFisher scientific). Cells were analyzed by flow cytometry using a BD LSRFortessa cell analyzer (BD Biosciences) and the Diva (BD Biosciences) and the FlowJo softwares.

In primary cell cultures, the expression of erythroid markers was monitored by flow cytometry using anti-CD36 (BD Horizon), anti-CD71 and anti-CD235a (BD PharMingen) antibodies and the proportion of enucleated RBCs was measured using the nuclear dye DRAQ5 (eBioscience). Flow cytometry analyses were performed using the Gallios analyzer and Kaluza software (Beckman-Coulter).

HPLC

HPLC analysis was performed using a NexeraX2 SIL-30AC chromatograph (Shimadzu) and the LC Solution software. Globin chains from differentiated HUDEP-2 cells (day 9) or from primary erythroid cells (day 16 of the in vitro erythroid differentiation) were separated by HPLC using a 250×4.6 mm, 3.6 μm Aeris Widepore column (Phenomenex). Samples were eluted with a gradient mixture of solution A (water/acetonitrile/trifluoroacetic acid, 95:5:0.1) and solution B (water/acetonitrile/trifluoroacetic acid, 5:95:0.1). The absorbance was measured at 220 nm.

Hemoglobin tetramers from mature RBCs (day 16 of the in vitro erythroid differentiation) were separated by CE-HPLC using a 2 cation-exchange column (PolyCAT A, PolyLC, Columbia). Samples were eluted with a gradient mixture of solution A (20 mM bis Tris, 2 mM KCN, pH, 6.5) and solution B (20 mM bis Tris, 2 mM KCN, 250 mM NaCl, pH, 6.8). The absorbance was measured at 415 nm.

Western Blot

RBCs from day 16 of the in vitro erythroid differentiation, were lysed for 30 min at 4° C. using a lysis buffer containing: 10 mM Tris, 1 mM EDTA, 0.5 mM EGTA, 1% Triton X-100, 0.1% SDS, 0.1% Na-deoxicholate, 140 mM NaCl (Sigma-Aldrich) and protease inhibitor cocktail (Roche-Diagnostics). Cell lysates were sonicated twice (50% amplitude, 10 sec per cycle, pulse 9 sec on/1 sec off) and underwent 3 cycles of freezing/thawing (3 min at −80° C./3 min at 37° C.). After centrifugation, the supernatant was collected and protein concentration was measured using the Pierce™ BCA Protein Assay Kit (ThermoScientific). After electrophoresis and protein transfer, γ- and α-globins were detected using the antibodies sc-21756 and sc-31110 (SantaCruz), respectively. The bands corresponding to γ- and α-globins were quantified using the Chemidoc and the Image lab Software (BioRad).

Results

Production of a Bifunctional LV for Gene Addition and Silencing

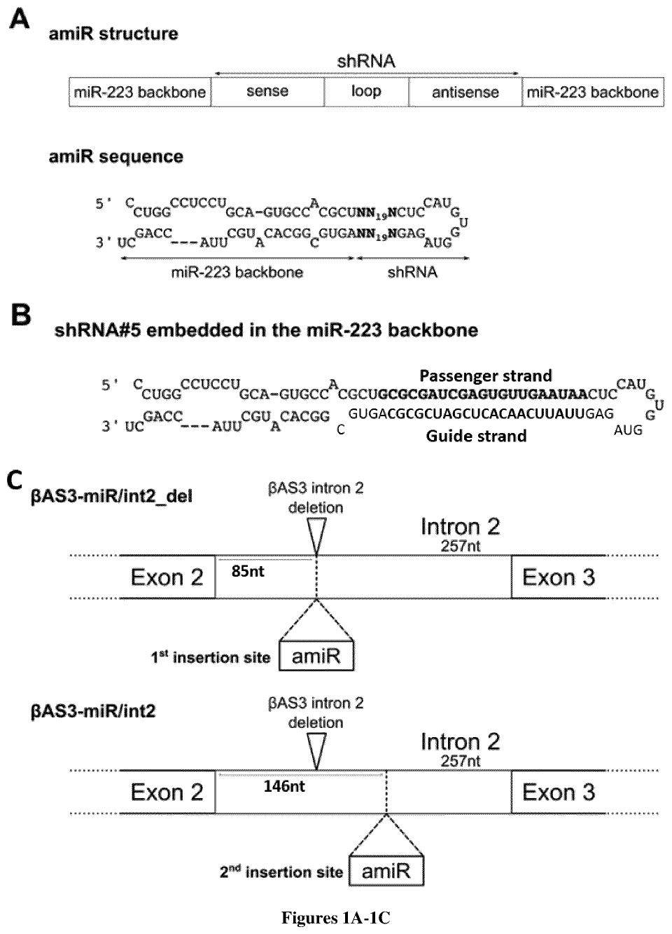

To re-express the HBG genes, we used an artificial microRNA (amiR) targeting BCL11A, described by Guda et al. (Guda et al., 2015) and Brendel et al. (Brendel et al., 2016). Briefly, this amiR is composed of the shRNA #5mod embedded in the miR-223 backbone (FIGS. 1A and 1B). This amiR targets the extra-large BCL11A isoform (BCL11A XL) responsible for HBG silencing (Liu et al., 2018; Trakarnsanga et al., 2014; Zhu et al., 2012). This strategy will avoid the potential side effects due to the silencing of other BCL11A isoforms. More precisely, the guide strand of this amiR targets the 3′ end of the coding sequence of BCL11A-XL mRNA (FIG. 1B). As in Guda's and Brendel's studies, we used the miR-223 backbone that has been extensively optimized to improve miRNA processing and reduce off-target binding by stringent strand selection (Amendola et al., 2009; Brendel et al., 2016; Guda et al., 2015).

Guda et al. (Guda et al., 2015) and Brendel et al. (Brendel et al., 2016) developed lentiviral vectors expressing an amiR targeting BCL11A to de-repress HBG. Compared to their studies, our approach is based on HBG de-repression through an amiR targeting BCL11A and the concomitant expression of the βAS3 transgene. This combined strategy will be more effective in providing therapeutic hemoglobin levels for both β-thalassemia and SCD.

Since amiR can be expressed using Pol II promoters (Amendola et al., 2009), we inserted our amiR in the second intron of the βAS3 transgene to express it under the control of the HBB promoter and 2 potent enhancers derived from the HBB locus control region (βAS3 LV; Weber et al., 2018), thus reducing potential amiR toxicity by limiting its expression to the erythroid lineage. Compared to the wild type intron of the HBB gene, βAS3 intron 2 carries a 593-bp deletion removing a region from 85 and 679 downstream of HBB exon 2. The total length of intron 2 is 257 nucleotides. The last 60 nucleotides of HBB intron 2 (which are retained in the βAS3 intron 2, nucleotides 198 to 257) are required for efficient 3′-end formation (Michael Antoniou et al., 1998).

To avoid negative effects on βAS3 RNA expression and processing (e.g. splicing and 3′end formation), we inserted the amiR between positions 85 and 86 or between 146 and 147 of the βAS3 intron 2 (βAS3-miR/int2_del and βAS3-miR/int2) because these regions are apparently not involved in RNA expression and splicing and far enough from the last 60 nucleotides to preserve 3′-end formation (FIG. 1C).

We generated 2 βAS3 LV-derived LVs containing the amiR in these two alternative positions (βAS3-miR/int2_del and βAS3-miR/int2). These LVs were tested in a human erythroid progenitor cell line (HUDEP-2; Kurita et al., 2013) and primary hematopoietic stem/progenitor cells (HSPCs) with the goal of achieving efficient BCL11A silencing without affecting βAS3 expression.

The Insertion of an amiR in βAS3 LV does not Affect Gene Transfer Efficiency

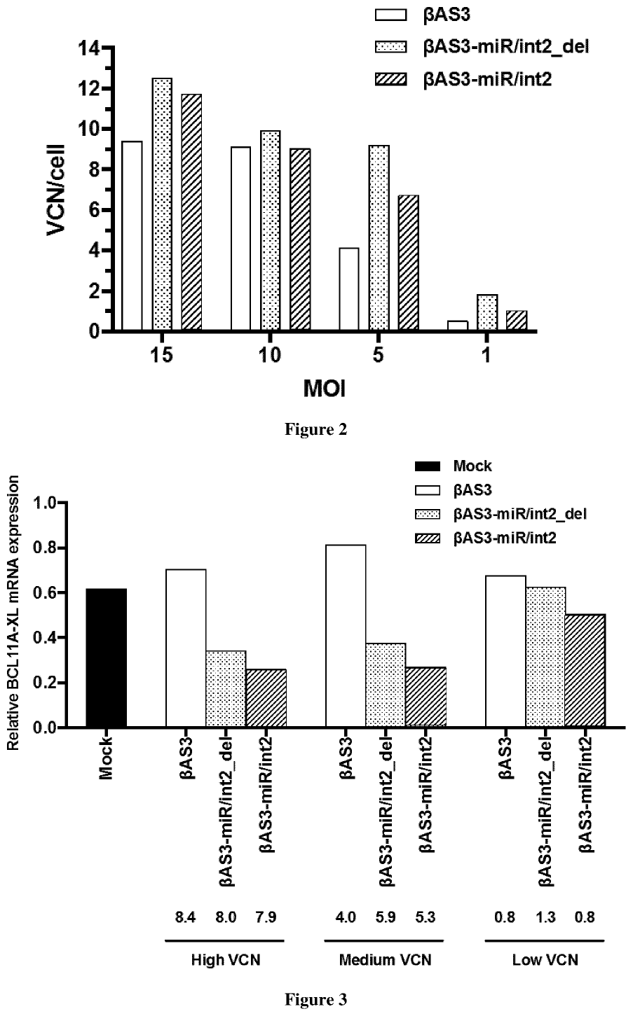

To assess the potential impact of the amiR on gene transfer efficiency, HUDEP-2 cells were transduced at increasing multiplicities of infection (MOI) with the different LV constructs: βAS3-miR/int2_del, βAS3-miR/int2 and the original LV containing only the βAS3 transgene (βAS3). Genomic DNA was extracted to measure the VCN per cell by ddPCR. Neither the insertion of the amiR, nor its position in intron 2 affected gene transfer efficiency (FIG. 2).

Bifunctional LVs Allow BCL11A-XL Silencing and βAS3 Transgene Expression

Mock- and LV-transduced HUDEP-2 cells were terminally differentiated into mature erythroblasts. We measured BCL11A-XL expression in mock- and LV-transduced HUDEP-2 cells. BCL11A-XL mRNA expression decreased in HUDEP-2 cells transduced with LVs containing the amiR (βAS3-miR/int2_del or βAS3-miR/int2) compared with control cells (mock-transduced or transduced with βAS3 LV) (FIG. 3). These results demonstrated that the amiR is expressed in the frame of the βAS3-expressing LVs and is able to reduce BCL11A-XL expression.

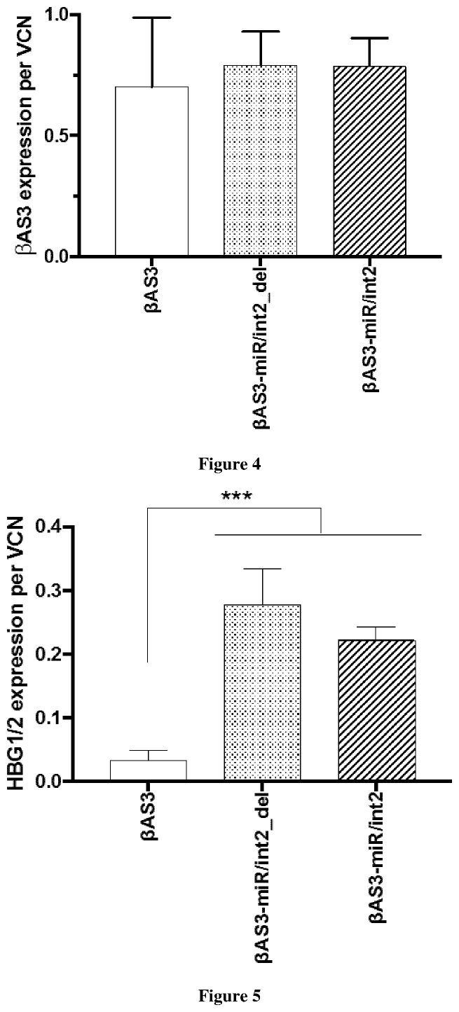

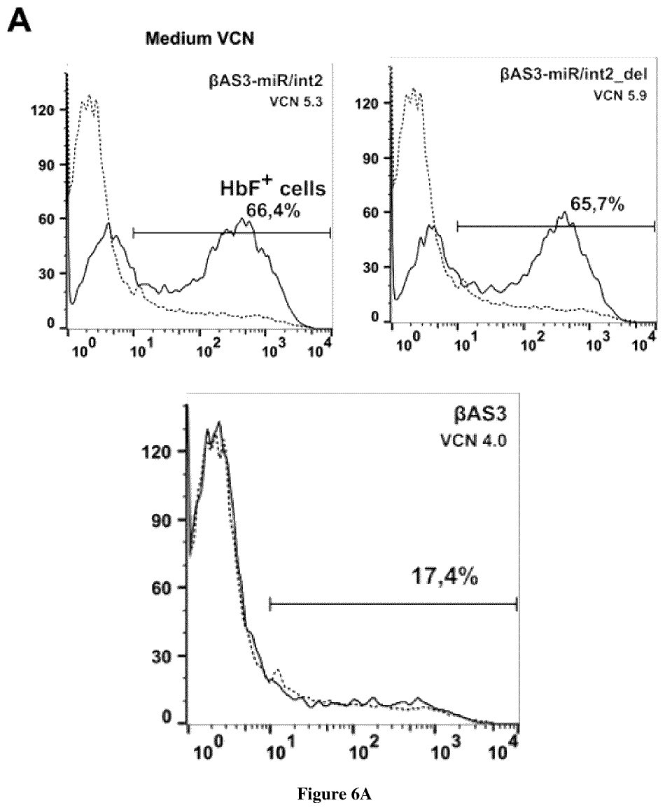

We then compared βAS3 transgene expression in HUDEP-2 cells transduced with βAS3-miR/int2_del, βAS3-miR/int2 and βAS3 LV. βAS3 transgene was expressed at similar levels for each LV (FIG. 4). Neither the insertion of the amiR nor its position in intron 2 affected βAS3 transgene expression.

amiR-Mediated BCL11A-XL Down-Regulation Induces HbF Re-Expression in HUDEP-2

To evaluate if BCL11A-XL silencing is associated with HBG re-activation, we measured HBG mRNA expression levels in terminally differentiated HUDEP-2. HBG expression was substantially higher in mature erythroblasts transduced with amiR-expressing LVs than in cells transduced with the βAS3 LV or in mock-transduced cells (FIG. 5). These results shows that amiR-mediated BCL11A-XL silencing leads to HBG gene re-activation.

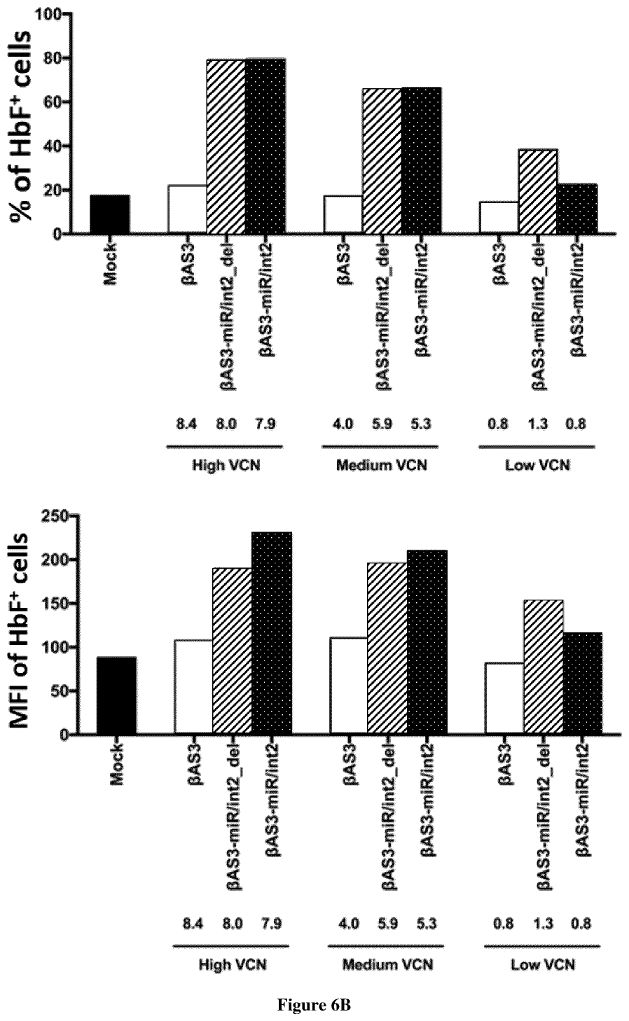

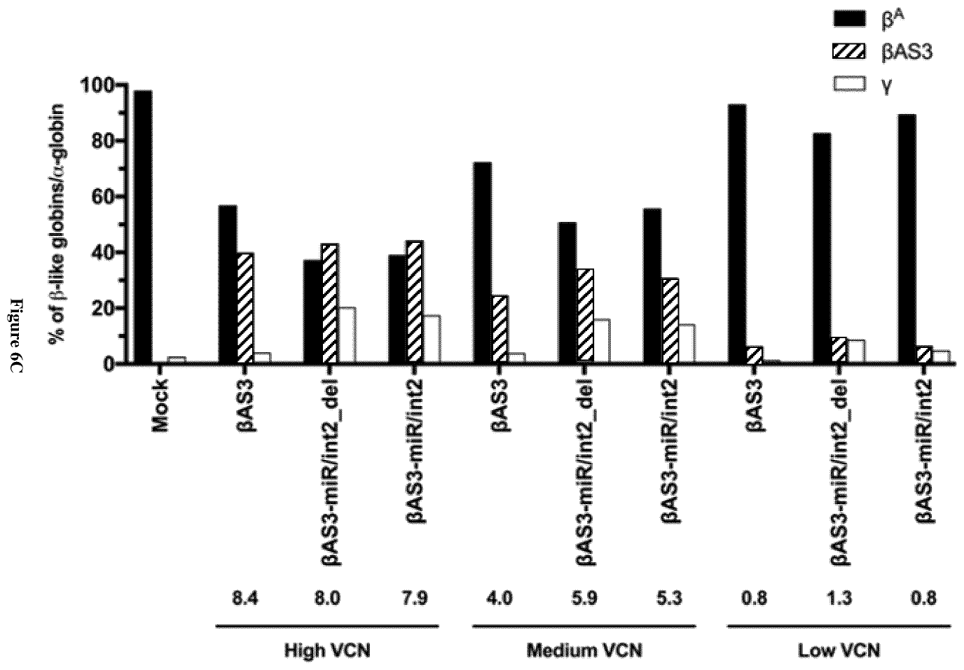

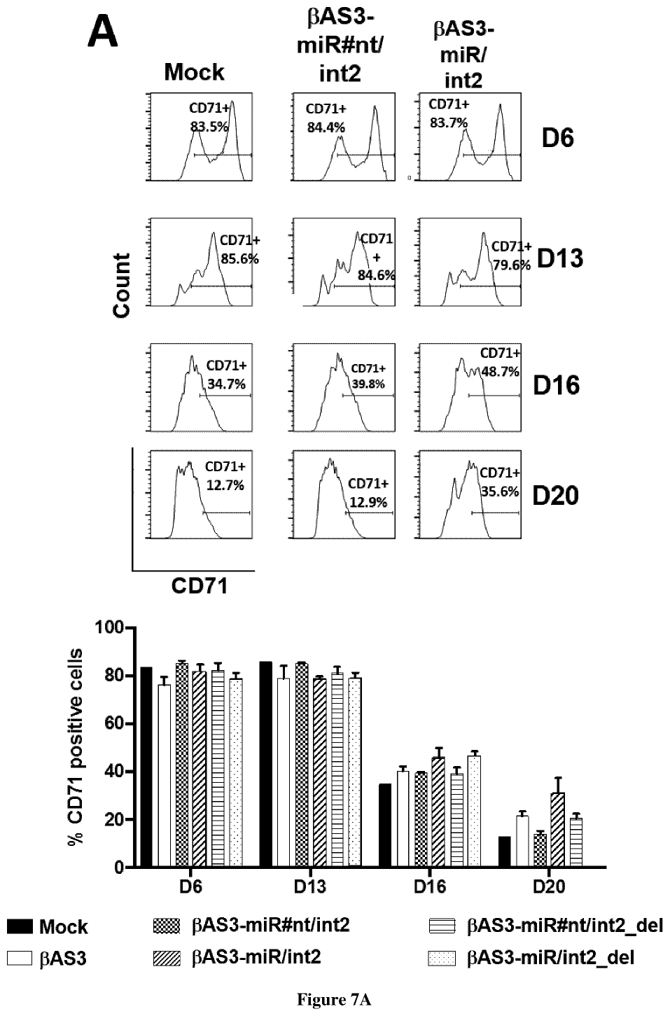

HbF expression was analyzed by flow cytometry in mock- and LV-transduced differentiated HUDEP-2 cells. Both the percentage of HbF populations and HbF content (measured as mean fluorescence intensity) were increased in samples transduced with LVs expressing the miR targeting BCL11A (FIGS. 6A, 6B and 6C). Reverse-phase HPLC analysis of single globin chains showed increased γ-globin expression upon BCL11A-XL silencing: overall the total amount of therapeutic β-like globin chains (γ+βAS3 globins) was higher in cells transduced with amiR-expressing LVs than in βAS3-transduced cells. Importantly, we observed a decrease in the levels of the endogenous adult β-globin (β^{A}) chains, which could further counteract RBC sickling in SCD.

Bifunctional LVs Induce HbF Re-Expression in Primary Erythroid Cells

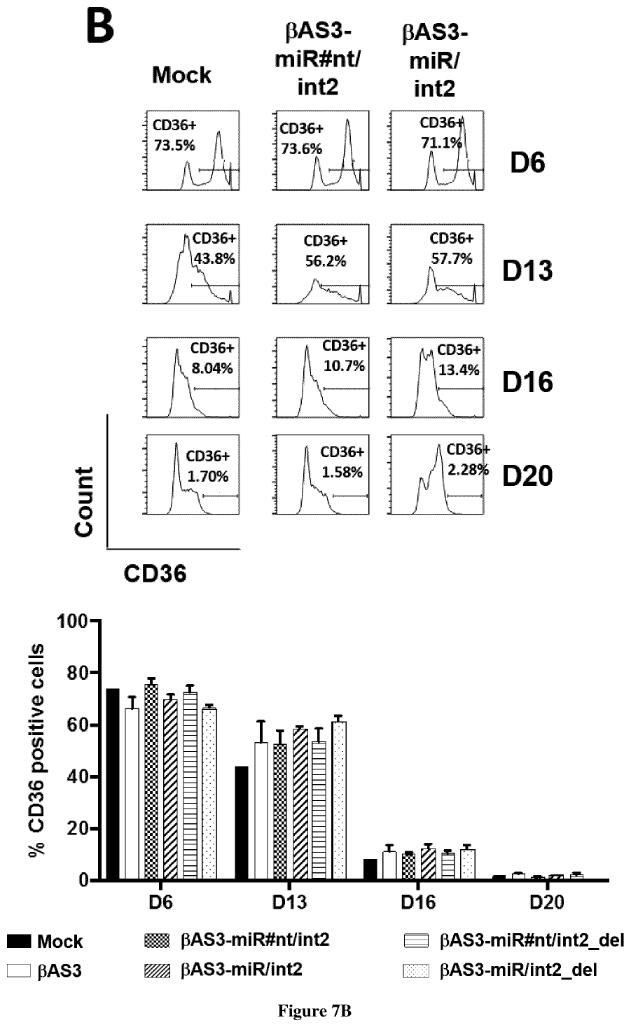

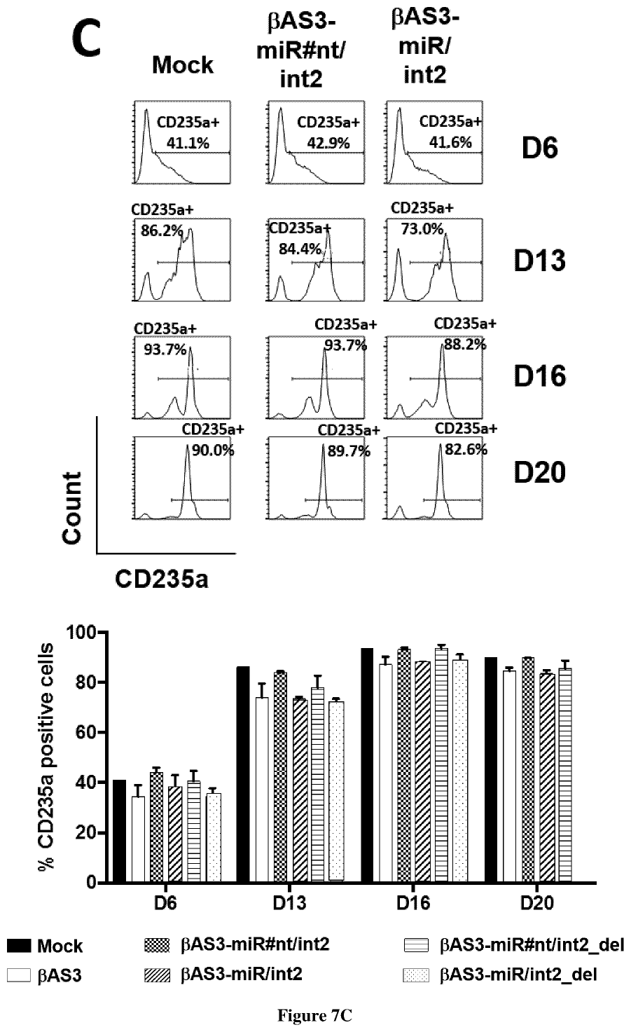

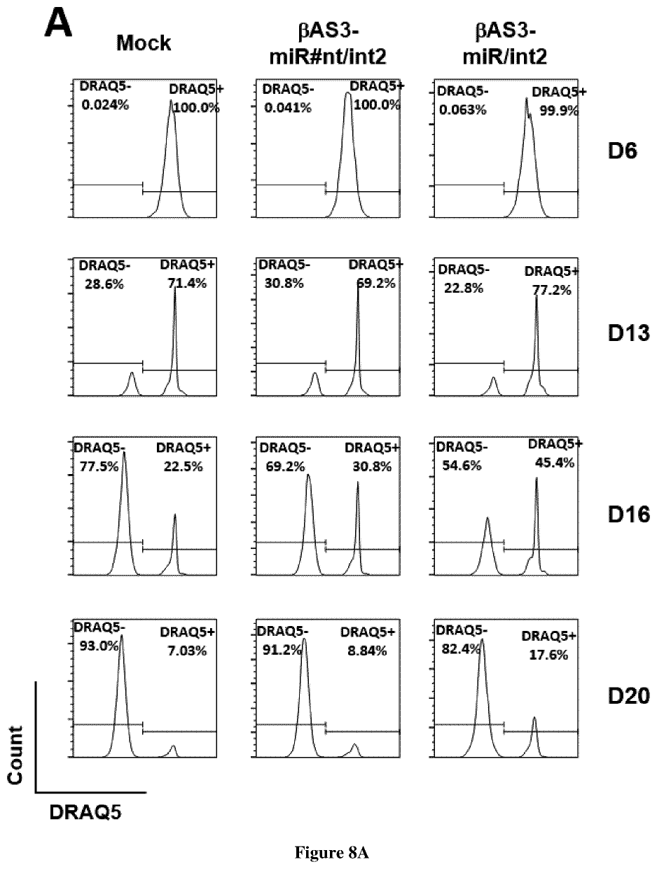

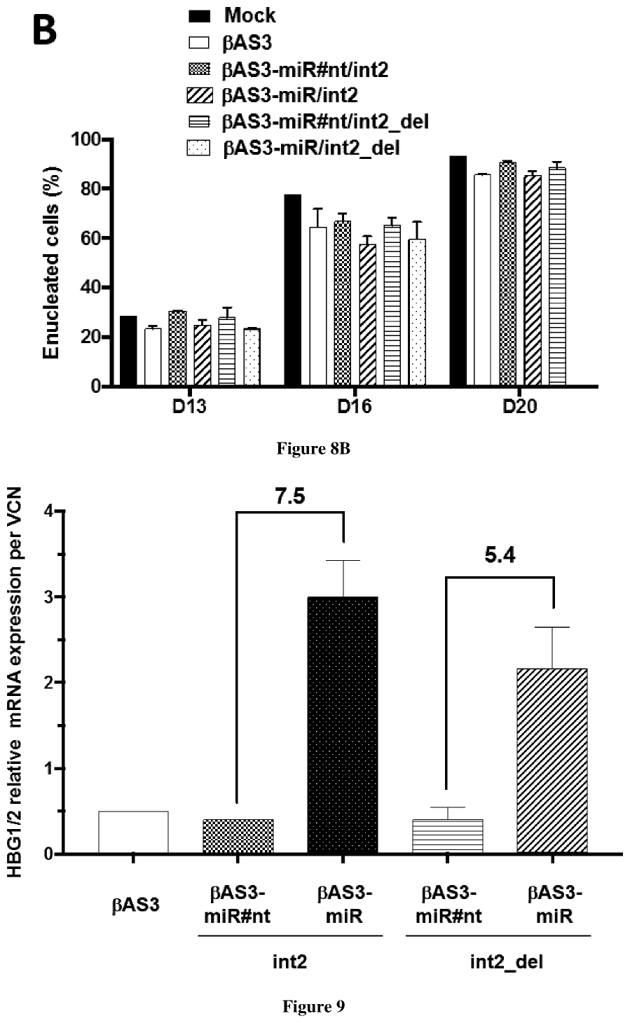

We transduced primary adult hematopoietic stem/progenitor cells (HSPCs) derived from healthy donors (HD) with bifunctional LVs harboring the amiR against BCL11A-XL. We introduced two new control LVs containing a non-targeting (nt) in the two different positions in intron 2 of the βAS3 transgene (βAS3-miR #nt/int2 and βAS3-miR #nt/int2_del). Mock- and transduced HSPCs were terminally differentiated into mature RBCs. Flow cytometry analysis of erythroid markers showed that erythroid differentiation was not altered upon HSPC transduction with bifunctional LVs (FIGS. 7A, 7B and 7C). Similarly, the proportion of enucleated RBCs along the differentiation was comparable between control and transduced samples with no impairment of enucleation upon expression of the amiR targeting BCLIIA-XL (FIGS. 8A and 8B).

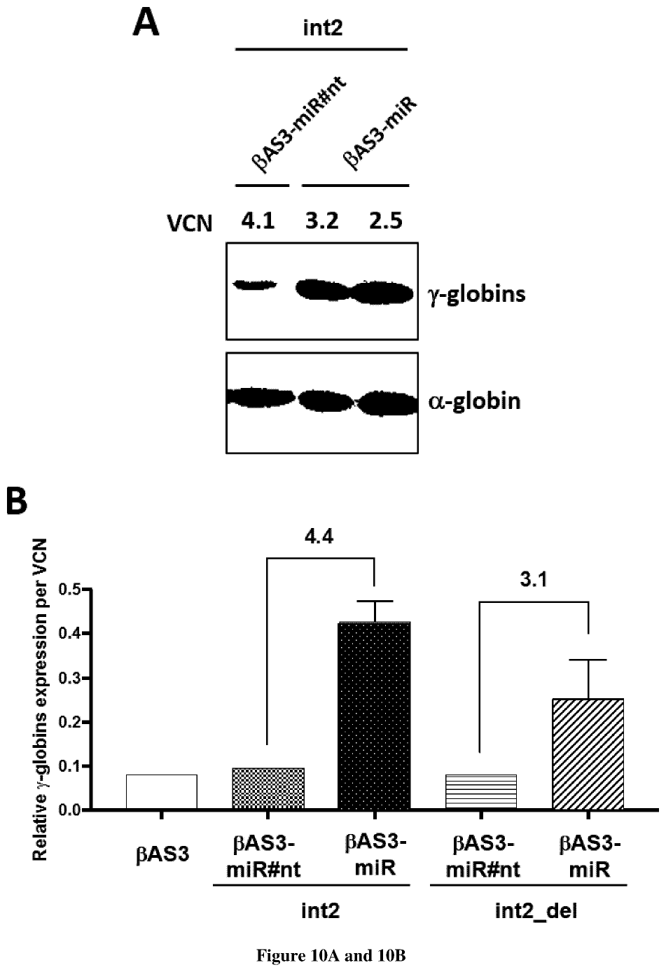

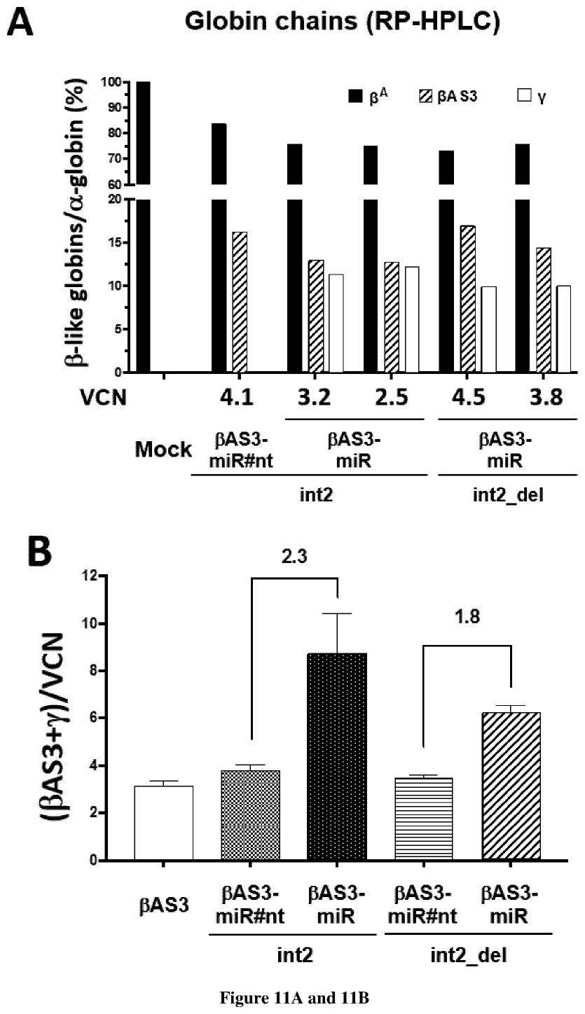

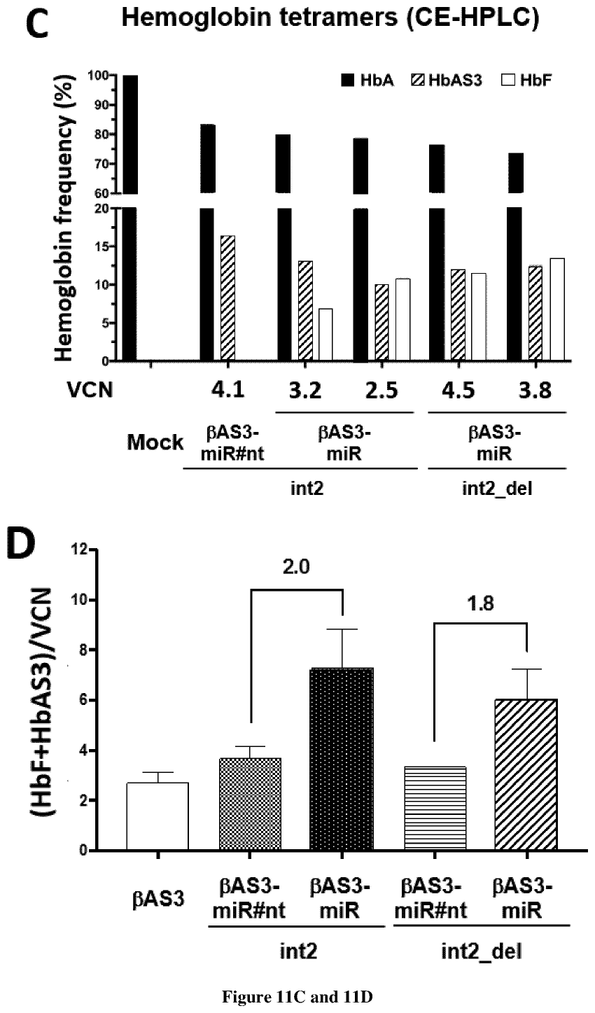

To evaluate the potential therapeutic effect of this strategy, we measured HBG mRNA expression in mock- and LV-transduced erythroid cells derived from HSPCs. HBG genes were de-repressed in cells transduced with LVs containing the amiR (βAS3-miR/int2_del or βAS3-miR/int2) compared to control cells (transduced with βAS3- or βAS3-miR #nt-LVs). Notably, we observed a 7.5-fold increase in HBG mRNA expression per VCN in cells transduced with the LV harboring the BCL11A-XL amiR in the int2 position (βAS3-miR/int2) (FIG. 9). De-repression of HBG1/2 genes was confirmed by Western Blot analysis: a 4.4-fold increase of γ-globin expression was observed in cells transduced with βAS3-miR/int2 compared to control cells transduced with βAS3-miR #nt/int2 (FIGS. 10A and 10B). γ-globin de-repression coupled with βAS3 expression resulted in a 2-fold increase in the total amount of therapeutic β-like globins and hemoglobins per VCN in RBCs derived from βAS3-miR-LV-compared to βAS-miR #ntLV-transduced HSPCs (FIGS. 11A, 11B, 11C and 11D).

### CONCLUSION

Overall, these results show that LVs expressing a βAS3 transgene and an amiR targeting BCL11A-XL could induce high-level of therapeutic globins. This combined strategy will likely be more effective than a classical gene addition approach to β-hemoglobinopathies.

### REFERENCES

Throughout this application, various references describe the state of the art to which this invention pertains. The disclosures of these references are hereby incorporated by reference into the present disclosure.
Amendola M, Passerini L, Pucci F, Gentner B, Bacchetta R, Naldini L. 2009. Regulated and Multiple miRNA and siRNA Delivery Into Primary Cells by a Lentiviral Platform. Mol Ther J Am Soc Gene Ther 17:1039-1052. doi:10.1038/mt.2009.48
Brendel C, Guda S, Renella R, Bauer D E, Canver M C, Kim Y-J, Heeney M M, Klatt D, Fogel J, Milsom M D, Orkin S H, Gregory R I, Williams D A. 2016. Lineage-specific BCL11A knockdown circumvents toxicities and reverses sickle phenotype. J Clin Invest 126:3868-3878. doi:10.1172/JCI87885
Guda S, Brendel C, Renella R, Du P, Bauer D E, Canver M C, Grenier J K, Grimson A W, Kamran S C, Thornton J, de Boer H, Root D E, Milsom M D, Orkin S H, Gregory R I, Williams D A. 2015. miRNA-embedded shRNAs for Lineage-specific BCL11A Knockdown and Hemoglobin F Induction. Mol Ther 23:1465-1474. doi:10.1038/mt.2015.113
Kurita R, Suda N, Sudo K, Miharada K, Hiroyama T, Miyoshi H, Tani K, Nakamura Y. 2013. Establishment of Immortalized Human Erythroid Progenitor Cell Lines Able to Produce Enucleated Red Blood Cells. PLOS ONE 8:e59890. doi:10.1371/journal.pone.0059890
Liu N, Hargreaves V V, Zhu Q, Kurland J V, Hong J, Kim W, Sher F, Macias-Trevino C, Rogers J M, Kurita R, Nakamura Y, Yuan G-C, Bauer D E, Xu J, Bulyk M L, Orkin S H. 2018. Direct Promoter Repression by BCL11A Controls the Fetal to Adult Hemoglobin Switch. Cell 173:430-442.e17. doi:10.1016/j.ce11.2018.03.016
Marktel S, Scaramuzza S, Cicalese M P, Giglio F, Galimberti S, Lidonnici M R, Calbi V, Assanelli A, Bernardo M E, Rossi C, Calabria A, Milani R, Gattillo S, Benedicenti F, Spinozzi G, Aprile A, Bergami A, Casiraghi M, Consiglieri G, Masera N, D'Angelo E, Mirra N, Origa R, Tartaglione I, Perrotta S, Winter R, Coppola M, Viarengo G, Santoleri L, Graziadei G, Gabaldo M, Valsecchi M G, Montini E, Naldini L, Cappellini M D, Ciceri F, Aiuti A, Ferrari G. 2019. Intrabone hematopoietic stem cell gene therapy for adult and pediatric patients affected by transfusion-dependent β-thalassemia. Nat Med 25:234-241. doi:10.1038/s41591-018-0301-6
Miccio A, Cesari R, Lotti F, Rossi C, Sanvito F, Ponzoni M, Routledge S J E, Chow C-M, Antoniou M N, Ferrari G. 2008. In vivo selection of genetically modified erythroblastic progenitors leads to long-term correction of β-thalassemia. Proc Natl Acad Sci 105:10547-10552. doi:10.1073/pnas.0711666105
Miccio A, Poletti V, Tiboni F, Rossi C, Antonelli A, Mavilio F, Ferrari G. 2011. The GATA1-H52 enhancer allows persistent and position-independent expression of a β-globin transgene. PloS One 6:e27955. doi:10.1371/journal.pone.0027955
Michael Antoniou, Geraghty F, Hurst J, Grosveld F. 1998. Efficient 3′-end formation of human β-globin mRNA in vivo requires sequences within the last intron but occurs independently of the splicing reaction 9.
Trakarnsanga K, Wilson M C, Lau W, Singleton B K, Parsons S F, Sakuntanaga P, Kurita R, Nakamura Y, Anstee D J, Frayne J. 2014. Induction of adult levels of β-globin in human erythroid cells that intrinsically express embryonic or fetal globin by transduction with KLF1 and BCL11A-XL. Haematologica 99:1677-1685. doi:10.3324/haematol.2014.110155
Weber L, Poletti V, Magrin E, Antoniani C, Martin S, Bayard C, Sadek H, Felix T, Meneghini V, Antoniou M N, El-Nemer W, Mavilio F, Cavazzana M, Andre-Schmutz I, Miccio A. 2018. An Optimized Lentiviral Vector Efficiently Corrects the Human Sickle Cell Disease Phenotype. Mol Ther Methods Clin Dev 10:268-280. doi:10.1016/j.omtm.2018.07.012
Zhu X, Wang Y, Pi W, Liu H, Wickrema A, Tuan D. 2012. NF-Y recruits both transcription activator and repressor to modulate tissue- and developmental stage-specific expression of human γ-globin gene. PloS One 7:e47175. doi:10.1371/journal.pone.0047175

1. A nucleic acid molecule having the sequence as set forth in SEQ ID NO:1 wherein a sequence encoding for an artificial microRNA (amiR) suitable for reducing the expression of BCL11A, is inserted i) between the nucleotide at position 85 and the nucleotide 86 at position in SEQ ID NO:1 and/or ii) between the nucleotide at position 146 and the nucleotide at position 147 in SEQ ID NO:1.

2. The nucleic acid molecule of claim 1 wherein the amiR comprises a shRNA that is embedded into a miRNA backbone.

3. The nucleic acid molecule of claim 2 wherein the miRNA backbone is derived from miR-142, miR-155, miR-181 and/or miR-223.

4. The nucleic acid molecule of claim 2 wherein the shRNA adopts a stem-loop structure wherein a stem region is formed by a guide strand and a passenger strand.

5. The nucleic acid molecule of claim 4 wherein the sequence encoding for the guide strand comprises the sequence as set forth in SEQ ID NO: 2.

6. The nucleic acid molecule of claim 4 wherein a loop segment is encoded by the sequence as set forth in SEQ ID NO:3.

7. The nucleic acid molecule of claim 2 wherein the sequence encoding for the shRNA comprises the sequence as set forth in SEQ ID NO:4.

8. The nucleic acid molecule of claim 1 wherein the sequence encoding for the amiR comprises the sequence as set forth in SEQ ID NO:5.

9. The nucleic acid molecule of claim 1 that has a sequence as set forth in SEQ ID NO:6 or SEQ ID NO:7.

10. A transgene encoding for an anti-sickling β-globin (HBB) wherein said transgene comprises the nucleic acid molecule of claim 1.

11. The transgene of claim 10 which comprises the sequence as set forth in SEQ ID NO:9 or SEQ ID NO:10.

12. The transgene of claim 10 which is placed under the transcriptional control of the HBB promoter and key regulatory elements from the 16-kb human β-locus control region (βLCR), wherein the key regulatory elements comprise the 2 DNase I hypersensitive sites HS2 and HS3.

13. A viral vector comprising the transgene of claim 10.

14. The viral vector of claim 13 which is a lentiviral vector.

15. A method of obtaining a population of host cells transduced with the transgene of claim 10, which comprises the step of transducing a population of host cells in vitro or ex vivo with the viral vector of claim 13.

16. The method of claim 15 wherein the host cell is selected from the group consisting of hematopoietic stem/progenitor cells, hematopoietic progenitor cells, hematopoietic stem cells (HSCs), pluripotent cells and induced pluripotent stem cells (iPS).

17. A method of treating a hemoglobinopathy in a subject in need thereof, comprising transplanting into the subject a therapeutically effective amount of the population of host cells obtained by the method of claim 16.

18. The nucleic acid molecule of claim 1, wherein the BCL11A is the BCL11A-XL isoform.

19. The method of claim 16 wherein the pluripotent cells are embryonic stem cells (ES).
